(19) United States
(12) Patent Application Publication
(10) Pub. No.: US 2024/0293456 A1
(43) Pub. Date: Sep. 5, 2024

(54) COMBINATIONS OF SMALL MOLECULE DRUG CONJUGATE AND CAR-EXPRESSING CYTOTOXIC LYMPHOCYTES AND METHODS OF TREATING CANCER USING THE SAME

(71) Applicant: PURDUE RESEARCH FOUNDATION, West Lafayette, IN (US)

(72) Inventors: Philip S. LOW, West Lafayette, IN (US); Weichuan LOU, Gaithersburg, MD (US)

(21) Appl. No.: 18/255,094

(22) PCT Filed: Nov. 29, 2021
(86) PCT No.: PCT/US2021/061026

§ 371 Date: May 30, 2023

Related U.S. Application Data
(60) Provisional application No. 63/119,234, filed on Nov. 30, 2020.

(51) Int. Cl. A61K 35/17; A61K 39/00; A61K 47/55; C07K 16/28

(52) U.S. Cl. CPC A61K 35/17; A61K 39/4611; A61K 39/4631; A61K 39/464412; A61K 47/551; C07K 16/2803; A61K 2239/13; A61K 2239/49

(57) ABSTRACT

A combination cancer therapy comprising a small molecule drug conjugate (SMDC), which targets a cell-surface receptor on an immunosuppressive cell or a cancerous cell, and cytotoxic lymphocytes, which express a chimeric antigen receptor (CAR); and a method of treating a patient for cancer using the same.

### PRIORITY

This application is related to and claims the priority benefit of U.S. Provisional Patent Application No. 63/199,234 filed Nov. 30, 2020. The content of the aforementioned application is hereby incorporated by reference in its entirety into this disclosure.

### TECHNICAL FIELD

The present disclosure relates to small molecule drug conjugates (SMDCs), chimeric antigen receptor (CAR)-expressing cytotoxic lymphocytes, compositions comprising a combination of the same, and methods of use thereof to treat cancer.

### BACKGROUND

Chimeric antigen receptors (CARs) are recombinant receptors that provide both antigen-binding and T cell activation functions. CAR-T cells have significant potential for treating cancers because of their tumor-specific activation and killing; for example, the CARs can allow the T cells to recognize a specific, pre-selected protein, or antigen, found on targeted tumor cells. CAR-T cells can be cultured and expanded in the laboratory, then re-infused into the autologous subject. Through the guidance of the engineered T cell receptor, CAR-T cells recognize and destroy the cancer cells that display the specific antigen on their surfaces.

Conventional CARs include a recognition region (e.g., a single chain fragment variable (scFv) region derived from an antibody) for recognition and binding to the antigen expressed by the tumor. The recognition region can be fused to the exoplasmic domain of a T cell receptor to enhance engagement of the T cell with the cancer cell. To facilitate rapid killing of the cancer cell, the CAR can be further modified to contain an activation signaling domain that, for example, can be derived from CD3 zeta chain (CD3ζ), a Fc receptor gamma signaling domain, or one or more costimulatory domains such as CD28, 4-1BB, ICOS, OX40, etc. An exemplary second-generation CAR consists of a scFv derived from an antibody for targeting, a CD3ζ for activating, a single cytoplasmic domain of a costimulatory receptor, such as CD28 or 4-1BB, and hinge and transmembrane domains.

Although CAR-T therapy's success in treating hematopoietic cancers is impressive, CAR-T therapies have been less effective on patients with solid tumors due in large part to an immunosuppressive tumor microenvironment (TME) that can inhibit the tumoricidal properties of T cells. In particular, the activities and survival of CAR-T cells in the TME are regulated by multiple immunosuppressive cells, including tumor-associated macrophages (TAMs), myeloid-derived suppressor cells (MDSCs), cancer-associated fibroblast (CAFs), tumor-associated neutrophils (TANs), and regulatory T cells (Tregs).

One of the significant challenges in killing solid tumors is caused by TAMs, which, along with MDSCs, are often prominent immune cells in the TME. TAMs and MDSCs, which can comprise up to at least 50% of the solid tumor mass, interact with cancer cells and other immune cells to facilitate tumor growth through promoting angiogenesis, immunosuppression, and inflammation. Further, both TAMs and MDSCs: (a) secrete immunosuppressive cytokines (e.g., interleukin 10 (IL-10) and transforming growth factor (TGF-0) and nitrosylate which inactivate T cell receptors, (b) upregulate immune checkpoint receptors, (c) promote the deposition of a dense extracellular matrix which can impede penetration of immune cells, and (d) produce immunosuppressive enzymes such as arginase 1, ecto-nuceloside triphosphate diphosphohydrolase (CD39), and 5′-nucleotidase. To enhance the performance of CAR-T cells in solid tumors, it is essential to inhibit the immunosuppressive activities of TAMs and MDSCs and/or convert TAMs and MDSCs in the TME from a tumor-supportive phenotype to less immunosuppressive and more tumoricidal phenotype.

In view of the above, it is an object to provide materials and methods to render TAMs and MDSCs in the TME tumoricidal. This and other objects and advantages, as well as inventive features, will become apparent from the detailed description provided herein.

### SUMMARY

A combination cancer therapy is provided. The combination comprises (a) at least one small molecule drug conjugate (SMDC) comprising a drug moiety conjugated to a ligand, wherein the ligand can be bound by a cell-surface receptor on an immunosuppressive cell or a cell-surface receptor on a cancerous cell (i.e., is specific to a receptor overexpressed on an immunosuppressive cell or a cancer cell), and (b) chimeric antigen receptor (CAR)-expressing cytotoxic lymphocytes, wherein the combination comprises a first amount of (a) and a second amount of (b), which together are effective to treat cancer.

The drug moiety in at least one SMDC is an agonist of a pattern recognition receptor located in the endosome or the cytoplasm of a cell, such as, for example, selected from the group consisting of an agonist of a toll-like receptor (TLR), an agonist of a phosphoinositide 3-kinase inhibitor (PI3Ki), an agonist of a stimulator of an interferon gene (STING), an agonist of a nucleotide-binding oligomerization domain (NOD)-like receptor (NLR), an agonist of a retinoic acid-inducible gene-I (RIG-I)-like receptor (RLR), an agonist of an absent in melanoma 2 (AIM2)-like receptor (ALR), an agonist of a receptor for advanced glycation end products (RAGE), an agonist of a kinase of the Pelle/interleukin-1 (IL-1) receptor-associated kinase (IRAK) family, such as an IRAK-M inhibitor, an inhibitor of Src homology 2 domain-containing tyrosine phosphatase 1 and 2 (SHP1/2), an inhibitor of T cell protein tyrosine phosphatase (TC-PTP), an inhibitor of diacylglycerol kinase (DGK), an inhibitor of enhancer of zeste homolog 2 (EZH2), and an inhibitor of transforming growth factor beta (TGFβ). The drug moiety in at least one SMDC can be a NF_{K}β activator or an I_{K}β kinase inhibitor. The drug moiety in at least one SMDC can be fluorescein isothiocyanate (FITC). In certain embodiments, the drug moiety in at least one SMDC is an agonist of TLR.

The ligand can comprise a folate receptor binding ligand or a fibroblast activation protein (FAP) ligand. The drug moiety and the ligand can be conjugated via a linker.

The linker can comprise a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol. The linker can be (PEG)₃.

In certain embodiments, the SMDC is a folate-TLR7 agonist, a releasable form of a folate-(PEG)₃-TLR7 agonist, or a non-releasable form of a folate-(PEG)₃-TLR7 agonist. In certain embodiments, the SMDC is a releasable form of a folate-(PEG)₃-TLR7 agonist.

The cytotoxic lymphocytes can be cytotoxic T cells, natural killer (NK) cells, lymphokine-activated killer (LAK) cells, or a combination of two or more of the foregoing.

In certain embodiments, the CAR is a fusion protein comprising a recognition region, a co-stimulation domain, and an activation signaling domain, and the CAR binds a cell-surface antigen on an immunosuppressive cell or a cancerous cell with high specificity. The recognition region can be a single chain variable fragment (scFv) of an antibody that binds to a cell-surface antigen with high specificity. The cell-surface antigen can be CD19. The co-stimulation domain can be CD28, CD137 (4-1BB), CD134 (OX40), or CD278 (ICOS). The activation signaling domain can be a T cell CD3 chain or a Fc receptor γ.

The recognition region can be a scFv region of an anti-FITC antibody, the co-stimulation domain can be CD28, and the activation signaling domain can be a T-cell CD3ζ chain. In certain embodiments, the recognition region can be a scFv region of an anti-CD19 antibody, the co-stimulation domain can be CD137 (4-lBB), and the activation signaling domain can be a T-cell CD3ζ chain. Still further, the recognition region can be a scFv region of an anti-CD19 antibody, the co-stimulation domain can be CD28, and the activation signaling domain can be a T-cell CD3ζ chain.

Also provided are methods of treating a subject (e.g., a patient) for cancer (e.g., in need thereof). The method comprises administering any of the combination cancer therapies or compounds described herein to the patient, whereupon the patient is treated for cancer. The lymphocytes can be autologous. Alternatively, the lymphocytes can be heterologous.

In certain embodiments, the cancer to be treated is a solid-tumor cancer. In certain embodiments, the cancer is a folate receptor expressing cancer.

In certain embodiments, the method of treating a subject for cancer further comprises imaging the subject (e.g., where the cancer is a solid-tumor cancer, imaging the tumor) prior to or during administering the combination cancer therapy. The administration step of the methods can further comprises administering a first therapeutically effective amount of at least one SMDC and a second therapeutically effective amount of the CAR-expressing cytotoxic lymphocytes (e.g., CAR-T cells). One or both of the at least one SMDC and the CAR-expressing cytotoxic lymphocytes can be administered to the subject via a mode of administration selected from the group consisting of intravenously, intramuscularly, intraperitoneally, and subcutaneously. In certain embodiments, the at least one SMDC and the CAR-expressing cytotoxic lymphocytes are administered to the subject via intravenous injection.

Further, in certain embodiments, the first therapeutically effective amount of the at least one SMDC and the second therapeutically effective amount of the CAR-expressing cytotoxic lymphocytes are administered simultaneously or sequentially (in either order).

In certain embodiments, the at least one SMDC is administered as a composition comprising one or more pharmaceutically acceptable carriers, adjuvants, diluents, excipients, vehicles, or a combination of any of the foregoing. In certain embodiments, the CAR-expressing cytotoxic lymphocytes can be administered as a composition comprising one or more pharmaceutically acceptable carriers, adjuvants, diluents, excipients, vehicles, or a combination of any of the foregoing.

The modes of administration of one or both of the at least one SMDC and/or the CAR-expressing cytotoxic lymphocytes can be selected from the group consisting of intravenously, intramuscularly, intraperitoneally, and subcutaneously. In certain embodiments, the mode of administration of the at least one SMDC is independent of the mode of administration of the CAR-expressing cytotoxic lymphocytes.

The methods can comprise a variety of dosing regimens. In certain embodiments, administering the at least one SMDC can comprise administering a dose (e.g., a therapeutically effective amount) of the at least one SMDC to the subject at least three times per week for a treatment period. In certain embodiments, administering the at least one SMDC can comprise administering a dose (e.g., a therapeutically effective amount) of the at least one SMDC to the subject at least four times per week for a treatment period. In certain embodiments, administering the at least one SMDC can comprise administering a dose (e.g., a therapeutically effective amount) of the at least one SMDC to the subject five or more times per week for a treatment period. In certain exemplary embodiments, administering the at least one SMDC can comprise administering a dose (e.g., a therapeutically effective amount) of the at least one SMDC to the subject five times per week for a treatment period.

In certain embodiments, the cancer to be treated by the method is a folate receptor α-expressing cancer or a folate receptor β-expressing cancer.

In certain embodiments of the method, administering the combination cancer therapy increases an amount of myeloid cells exhibiting an immune-stimulating phenotype in a tumor microenvironment (TME) of the subject as compared to an amount of myeloid cells exhibiting an immunosuppressive phenotype in the TME. In certain embodiments of the method, administering the combination cancer therapy reduces an amount of myeloid-derived suppressor cells present within a TME of the subject.

Combination cancer therapies are also provided that comprise a first pharmaceutical composition and a second pharmaceutical composition, wherein the combination comprises a first amount of the first pharmaceutical composition and a second amount of the second pharmaceutical composition. The first pharmaceutical composition can comprise at least one SMDC comprising a drug moiety or a pharmaceutically acceptable salt thereof conjugated to a ligand, wherein the ligand is specific to a receptor overexpressed on an immunosuppressive cell or a cancer cell. The second pharmaceutical composition can comprise CAR-expressing cytotoxic lymphocytes. In certain embodiments, the combination comprises a first amount of the first pharmaceutical composition and a second amount of the second pharmaceutical composition.

In certain embodiments, the first pharmaceutical composition comprises one or more pharmaceutically acceptable carriers, adjuvants, diluents, excipients, vehicles, or a combination of any of the foregoing. In certain embodiments, the second pharmaceutical composition comprises one or more pharmaceutically acceptable carriers, adjuvants, diluents, excipients, vehicles, or a combination of any of the foregoing.

### BRIEF DESCRIPTION OF THE FIGURES

The embodiments and other features, advantages, and aspects, and the matter of attaining them, will become apparent in light of the following detailed description of various examplary embodiments. The detailed description will be better understood when taken in conjunction with the accompanying drawings.

FIG. 1 shows the structure of releasable (FA-PEG₃-(R) TLR7-1A; labeled Compound 1) and non-releasable (FA-PEG₃-(NR) TLR7-1A; labeled Compound 2) forms of a folate-TLR7 agonist.

FIGS. 2A-2C are flow cytometry histograms of expression level of folate receptor, TLR7, and mouse CD19 vs. percent of maximum (Max), which show the selection of a CD19⁺ cancer cell line that expresses no folate nor TLR7 receptor. FIG. 2A shows that all CT26, 4T1 and EMT6 cells were found to lack detectable FR (as compared to the positive control in L1210A cells), whereas FIG. 2B shows that none of the cells expressed TLR7 (as compared with the positive control in 24JK cells). FIG. 2C shows each cell line transduced with murine CD19 (mCD19) using a lentiviral vector (4T1 cells expressing mCD19 labeled 4T1-mCD19, CT26 cells expressing mCD19 labeled CT26-mCD19, and EMT6 cells expressing mCD19 labeled EMT6-mCD19) and sorted for cells that expressed high levels of CD19.

FIG. 3A are flow cytometry dot plots of anti-murine CD19 CAR-T cells vs. SSC-A (10\hat{ }³), which show the expression of anti-murine CD19 scFv on transduced and non-transduced, murine T cells as measured by flow cytometry using anti-rat-Alexa 594 antibody for staining.

FIG. 3B is a graph of 4T1-mCD19 cells vs. % cytotoxicity against mouse anti-mCD19 CAR-T cells, which shows the results of an assay to determine whether the anti-mCD19 CAR-T cells were cytotoxic to the 4T1-mCD19⁺ cancer cells.

FIGS. 4A and 4B show graphs of in vivo tumor size (mm³) as compared to days of treatment of either no treatment (control), treatment with CAR-T cells and PBS, or treatment with a combination therapy comprising CAR-T cells and 3 nmol non-releasable FA-PEG₃-(NR) TLR7-1A conjugate (FA-TLR7-1A; Compound 2) administered 5 times/week, with FIG. 4A displaying results for heterogeneous 4T1-mCD19 cohorts, and FIG. 4B displaying results for homogenous 4T1-mCD19 cohorts.

FIG. 4C show graphs representative of tumor size and body weight of the mice over various days following rechallenge of the cured mice administered at least 30 days following the initial administration of the combination therapy.

FIGS. 5A and 5B show graphs representative of the loss of CD19 expression for CAR-T treated groups, with FIG. 5A showing data related to a heterogenous tumor and FIG. 5B showing data related to a single cell clone tumor.

FIG. 6A shows a representation of the various dosing frequencies of FA-TLR7-1A evaluated.

FIG. 6B shows a graphical representation of the tumor size versus days after treatment with dosage frequency varied.

FIGS. 7A-7C show graphical representations of the effect of tumor size on the impact of the combination therapies hereof.

FIGS. 8A-8C show graphical representations of data related to a tumor microenvironment phenotype following administration of embodiments of the combination therapies described herein, where FIG. 8A is a graph of treatment vs. iNOS⁺/arginasel⁺ in F4/80⁺, which shows the M1/M2 (iNOS⁺/arginasel⁺) macrophage ratio in the tumor after treatment with CAR-T cells only or the combination of CAR-T cells and the non-releasable FA-TLR7-1A (Compound 2) as compared to no treatment, FIG. 8B is a graph of treatment vs. F4/80⁺ percentage in tumor, which shows the percentage of total macrophages in the tumor after treatment with CAR-T cells only or the combination of CAR-T cells and Compound 2 as compared to no treatment, and FIG. 8C is a graph of treatment versus percentage CD11b⁺Gr-1⁺ cells in tumor, which shows the percentage of total myeloid-derived stem cells (MDSCs) in the tumor after treatment with CAR-T cells only or the combination of CAR-T cells and Compound 2 as compared to no treatment.

FIGS. 9A and 9B show graphical data representative of (a) the M1/M2 phenotypic ratio in the spleen following administration of a certain embodiment of the combination therapy hereof (FIG. 9A), and (b) percentage of macrophages present in the spleen following administration of a certain embodiment of the combination therapy hereof (FIG. 9B).

FIGS. 10A-10F show graphical data related to the number of infiltrated total and activated T cells and CAR-T cells inside a tumor following the administration of various treatments described herein.

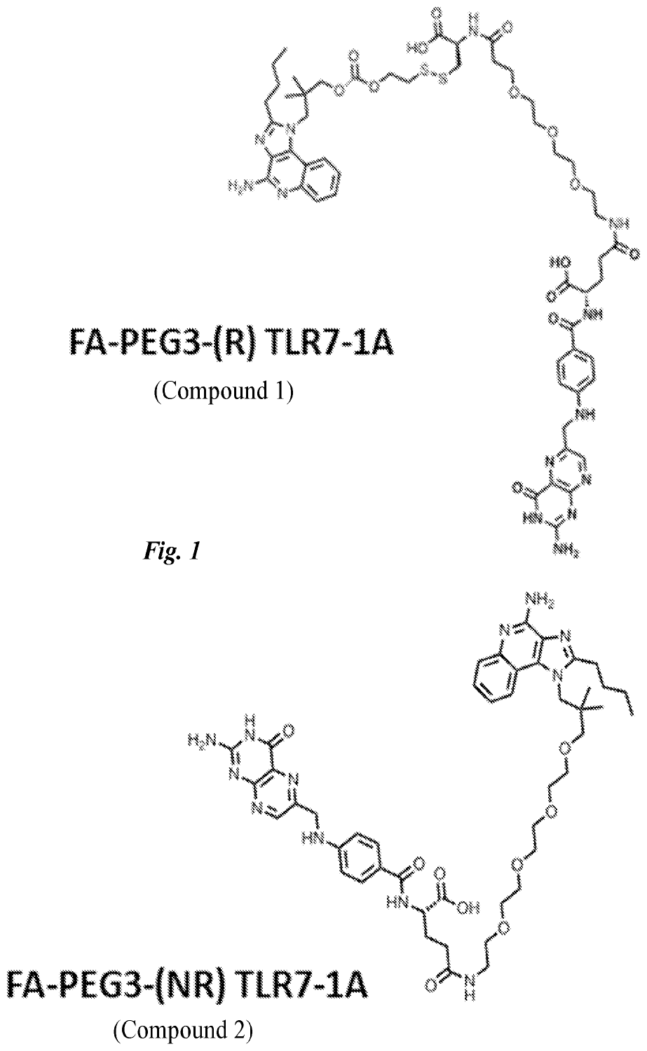

Wherever feasible and convenient, like reference numerals are used in the figures and the description to refer to the same or like parts or steps. The drawings are in a simplified form and not to precise scale. While the present disclosure is susceptible to various modifications and alternative forms, exemplary embodiments thereof are shown in the drawings and are described in detail.

### DETAILED DESCRIPTION

This disclosure is predicated on the discovery of a way to utilize an agonist of a pattern recognition receptor located in the endosome or the cytoplasm of a cell, such as an agonist of a toll-like receptor (TLR), and in particular TLR7, to convert tumor-associated macrophages (TAMs) and myeloid-derived suppressor cells (MDSCs) from an anti-inflammatory (M2-like) phenotype to a proinflammatory (M1-like) phenotype, while minimizing, if not eliminating, toxicity associated with systemic injection of the agonist by itself (e.g., off-target toxicity).

Generally, the combinations, compositions, and methods hereof employ at least one small molecule drug conjugate (SMDC) comprising a drug moiety (e.g., an agonist) conjugated to a ligand. In certain embodiments, the ligand binds with specificity to a cell-surface receptor on folate receptor beta (FRβ)-expressing myeloid cells, which in tumor-bearing mammals are predominantly immunosuppressive and almost exclusively located within a tumor microenvironment (TME). Upon uptake by the targeted cells, the drug moiety of the SMDC can bind a TLR and initiate signaling events to reprogram the cells into a more immune-stimulating phenotype (e.g., M1-like). Administration of the SMDC can additionally be combined with the administration of chimeric antigen receptor (CAR)-expressing cytotoxic lymphocytes to augment the potency of the CAR cell therapy with little to no off-target toxicity observed.

While, in certain instances TLR agonists have been found effective at repolarizing TAMs and MDSCs, TLR agonists can be too toxic for systemic administration. Moreover, treatment with a TLR agonist alone pursuant to conventional techniques has shown moderate anti-tumor efficacy at best.

Accordingly, the present combinations, compositions, and methods provide for a cancer treatment that is not only effective against solid tumors, but also selectively targets the agonist to a receptor on the TAMs and/or MDSCs inside the cancerous tumor such that systemic and/or off-target toxicity is avoided. Additionally, the TLR agonist can modify certain properties of other infiltrating immune cells, including CAR-T cells and normal T cells, thereby significantly augmenting the potencies of CAR therapies administered in combination therewith.

The term “off-target toxicity” means organ or tissue damage or a reduction in the subject's weight that is not desirable to the physician or other individual treating the subject, or any other effect on the subject that is a potential adverse indicator to the treating physician (e.g., B cell aplasia, a fever, a drop in blood pressure, or pulmonary edema). The terms “treat,” “treating,” “treated,” or “treatment” (with respect to a disease or condition) is an approach for obtaining beneficial or desired results including and preferably clinical results and can include, but is not limited to, one or more of the following: improving a condition associated with a disease, curing a disease, lessening severity of a disease, increasing the quality of life of one suffering from a disease, prolonging survival and/or a prophylactic or preventative treatment. In reference to cancer, in particular, the terms “treat,” “treating,” “treated,” or “treatment” can additionally mean reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, “CAR therapy” and “CAR-T cells” refer to a cytotoxic lymphocyte cell (e.g., a T cell) or population thereof that has been modified through molecular biological methods to express a CAR on the cell surface. The CAR is a polypeptide having a pre-defined binding specificity to a desired target and is operably connected to (e.g., as a fusion, separate chains linked by one or more disulfide bonds, etc.) the intracellular part of a cell activation domain. By bypassing MHC class I and class II restriction, CAR engineered lymphocyte cells of both CD8+ and CD4+ subsets can be recruited for redirected target cell recognition.

The CARs comprise a recognition region as is further defined herein. In certain embodiments, a CAR can additionally include an activation signaling domain that, for example, can be derived from a T cell CD3-zeta (CD3ζ) chain, a Fc receptor gamma signaling domain or a Fc receptor γ, or one or more costimulatory domains such as CD28, CD137 (4-1BB), CD278 (ICOS), or CD134 (OX40).

Certain CARs are fusions of binding functionality (e.g., as a single-chain variable fragment (scFv) derived from a monoclonal antibody) to CD3ζ transmembrane and endodomain. Such molecules result in the transmission of a zeta signal in response to recognition by the recognition receptor binding functionality of its target. There are, however, many alternatives. By way of non-limiting example, an antigen recognition domain from native T cell receptor (TCR) alpha and beta single chains can be used as the binding functionality. Alternatively, receptor ectodomains (e.g., CD4 ectodomain) can be employed. All that is required of the binding functionality is that it can bind a given target with high affinity in a specific manner.

Additionally, “binds with specificity,” “binds with high affinity,” or “specifically” or “selectively” binds, when referring to a ligand/receptor, a recognition region/targeting moiety, a nucleic acid/complementary nucleic acid, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a typical embodiment, a molecule that specifically binds a target will have an affinity that is at least about 10⁶ liters/mol (K_{σ}=10^{˜6} M), and preferably at least about 10 liters/mol, as determined, for example, by Scatchard analysis. It is recognized by one of skill in the art that some binding compounds can specifically bind to more than one target, for example an antibody specifically binds to its antigen, to lectins by way of the antibody's oligosaccharide, and/or to an Fc receptor by way of the antibody's Fc region.

The combinations, compositions, and methods will now be described in detail. For the purposes of promoting an understanding of the principles presented herein, reference is made to the embodiments illustrated in the drawings and specific language is used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims.

As noted above, the combinations, compositions and methods of the present disclosure employ at least one SMDC in combination with administration of CAR therapy. In certain embodiments, the SMDC comprises a drug moiety (e.g., the agonist) conjugated to a ligand (e.g., a targeting moiety), such as by way of a linker. For example, in certain embodiments a TLR agonist (the drug moiety) can be linked to folic acid (the ligand) to result in a folate-TLRa conjugate (FA-TLRa).

The ligand of the SMDC can bind a cell-surface receptor (e.g., FRβ or fibroblast activation protein (FAP)) on an immunosuppressive cell. Upon uptake of the conjugate by the targeted cells, such as an activated macrophage (e.g., a TAM or a MDSC) or a cell-surface receptor on a cancerous cell, the conjugate can bind a TLR and initiate signaling events that reprogram the TAMs/MDSCs into a more immune-stimulating phenotype (i.e., M1-like).

The administration of the SMDC is combined with the administration of CAR-expressing cytotoxic lymphocytes, which prevents the inactivation of such CAR-expressing lymphocytes in the TME that has been observed with conventional approaches. In certain embodiments, the SMDC targets immunosuppressive cells (and/or cancerous cells) in the tumor and delivers the drug moiety to the targeted cells, thereby enhancing the infiltration and activities of the CAR-expressing cytotoxic lymphocytes within the TME while also avoiding systemic toxicity. Administration of a SMDC, along with the CAR-expressing cytotoxic lymphocytes, results in better cytotoxicity against cancer cells in solid tumors than CAR-expressing cytotoxic lymphocytes alone.

Thus, a combination cancer therapy is provided. In certain embodiments, the combination comprises (a) at least one SMDC, which comprises a drug moiety conjugated to a ligand (e.g., targeting moiety), which can be bound by a cell-surface receptor on an immunosuppressive cell or a cell-surface receptor on a cancerous cell, and (b) CAR-expressing cytotoxic lymphocytes, wherein the combination comprises a first amount of (a) and a second amount of (b), which together are therapeutically effective to treat cancer.

### SMDC

In at least one embodiment, the at least one SMDC of the combination therapy comprises a drug moiety. Any therapeutic agent (referred to herein as a “drug”) that can reprogram activated macrophages with an M2 or M2-like phenotype to an M1 or M1-like phenotype can be used. The drug can operate in the endosome and/or the cytoplasm of the cell (e.g., depending on its structure). In at least one embodiment, the drug in at least one SMDC comprises an immunomodulatory compound (e.g., a drug that positively controls a pattern recognition receptor and/or its downstream signaling pathways (part of the innate immune system)), such as, for example, an agonist of a pattern recognition receptor located in the endosome or the cytoplasm of a cell, such as an agonist of a TLR, a stimulator of an interferon gene (STING), a nucleotide-binding oligomerization domain (NOD)-like receptor (NLR), a retinoic acid-inducible gene-I (RIG-I)-like receptor (RLR), absent in melanoma 2 (AIM2)-like receptor (ALR), a receptor for advanced glycation end products (RAGE), a kinase of the Pelle/interleukin-1 (IL-1) receptor-associated kinase (IRAK) family, such as an IRAK-M inhibitor, an inhibitor of Src homology 2 domain-containing tyrosine phosphatase 1 and 2 (SHP1/2), an inhibitor of T cell protein tyrosine phosphatase (TC-PTP), an inhibitor of diacylglycerol kinase (DGK), an inhibitor of enhancer of zeste homolog 2 (EZH2), or an inhibitor of transforming growth factor beta (TGFβ). In other embodiments, the drug in at least one SMDC comprises a phosphoinositide 3-kinase inhibitor (PI3Ki) or other inhibitor that negatively controls the adaptive immune system (e.g., which may be employed alone or in conjunction with an immune modulator that targets a pattern recognition receptor). The drug moiety in at least one SMDC can be a nuclear factor kappa-light-chain-enhancer of activated B cells (NF_{K}β) activator or an activator of 1-kappa-0 (I_{K}β) kinase inhibitor (as shown in Table 1, for example). The drug moiety in at least one SMDC can be an imaging agent, such as a radiolabel or an optical label, such as a fluorescent label, of which fluorescein isothiocyanate (FITC) is an example.

TABLE 1
NFκβ Activators/Inducers

| Compound | Structure |
|---|---|
| AA | 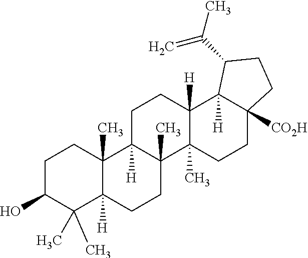 |
| BB | 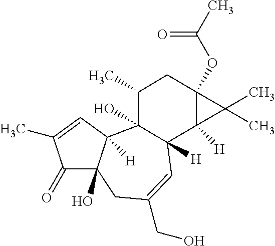 |
| CC | 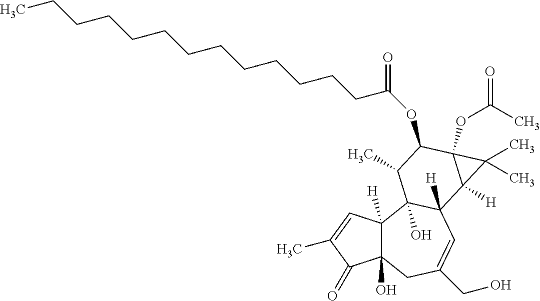 |
| DD | 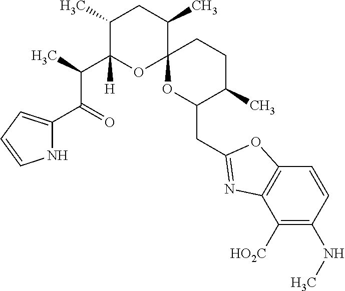 |
| EE | 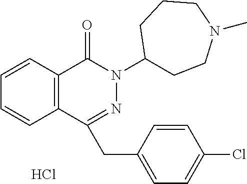 [SMILES: CN1CCCC(n2nc(Cc3ccc(Cl)cc3)c3ccccc3c2=O)CC1 ; Cl] |
| FF | 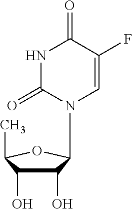 |
| GG | 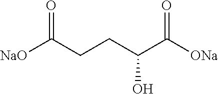 |

“Toll-like receptors” or “TLRs” are a class of proteins that play a role in the innate immune system and are an example of pattern recognition receptors. TLRs can be single, membrane-spanning receptors that recognize structurally conserved molecules derived from microbes. TLRs can be expressed on the membranes of leukocytes including, for example, dendritic cells, macrophages, natural killer cells, cells of adaptive immunity (e.g., T and B lymphocytes) and non-immune cells (epithelial and endothelial cells and fibroblasts). Non-limiting examples of TLRs include TLR1, TLR2, TLR3, TLR4, TLR5, TLR6, TLR7, TLR8, TLR9, TLR10, TLR11, TLR12, and TLR13. In some embodiments, a TLR agonist provided herein binds to one or more TLR. In some embodiments, a TLR agonist provided herein binds to TLR7, TLR8, or TLR9. In some embodiments, a TLR agonist provided herein binds to TLR7. In some embodiments, a TLR agonist provided herein binds to TLR7 and TLR8. In some embodiments, an agonist is a ligand that binds to and activates a receptor.

In the combinations, compositions and methods hereof, any suitable immunomodulatory (e.g., immunostimulatory) small molecule that binds to a TLR can be used as the drug moiety. Non-limiting examples of TLR agonists include a TLR 7 agonist, a TLR8 agonist, and a TLR7/8 agonist, such as:

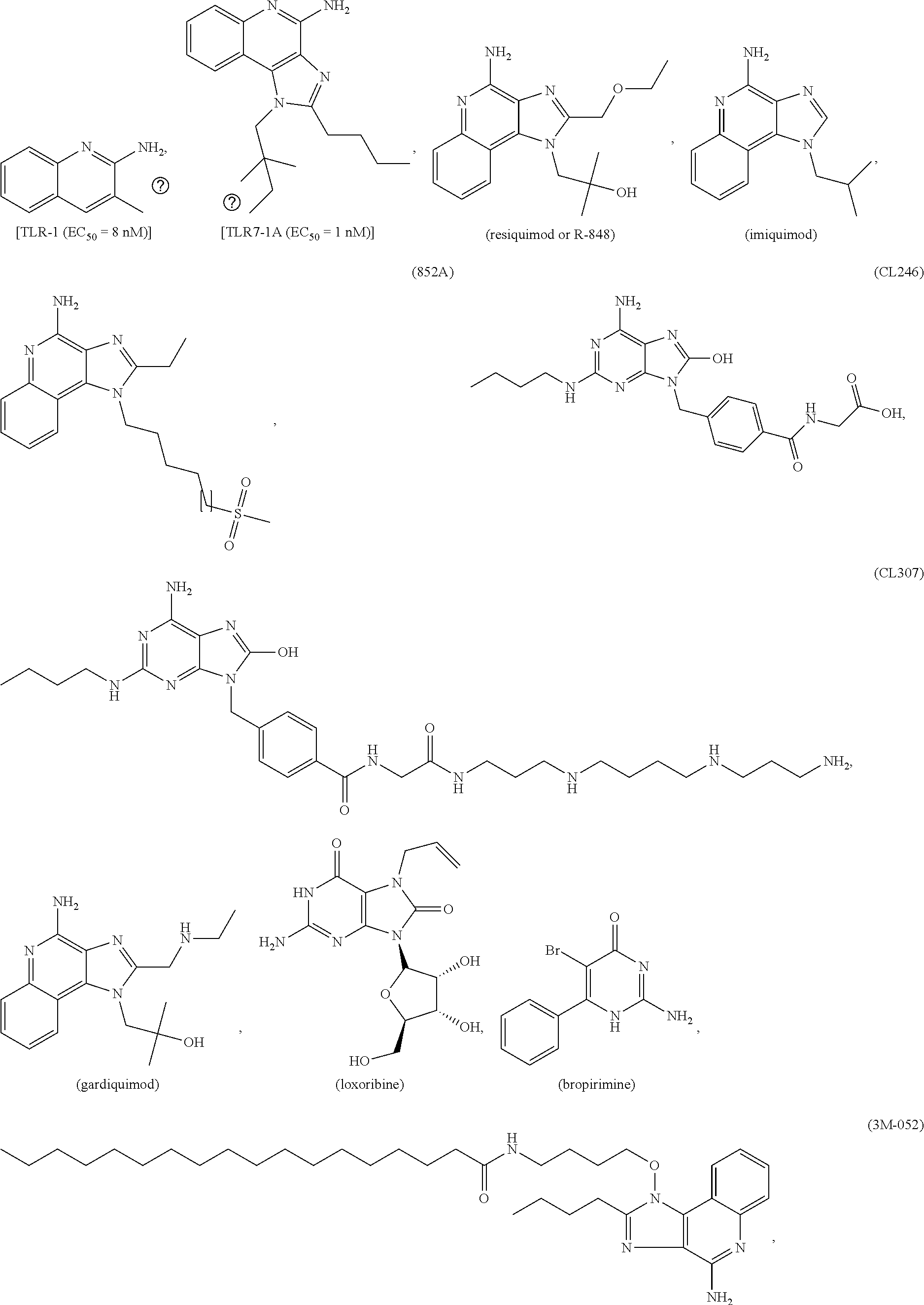

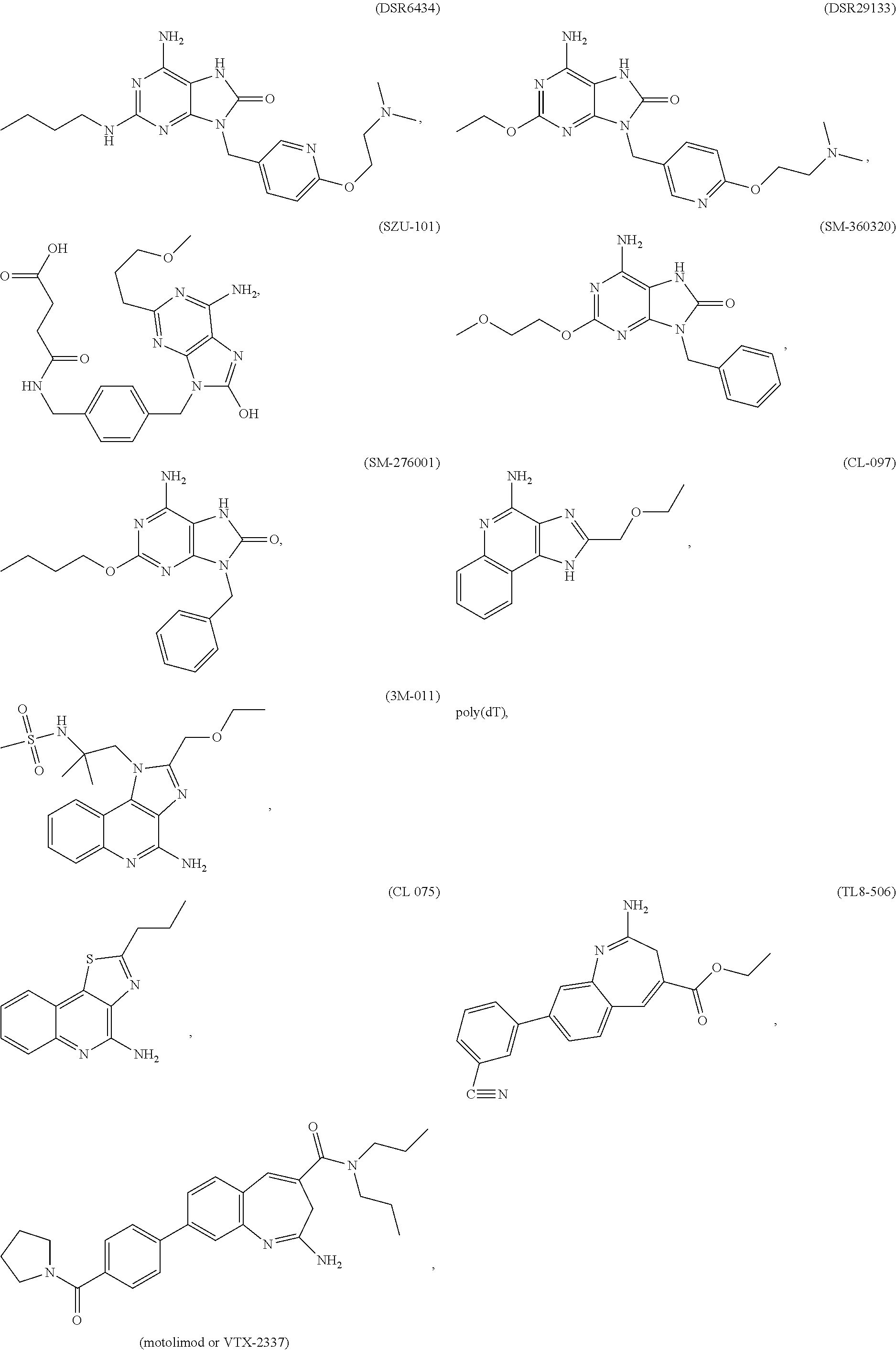

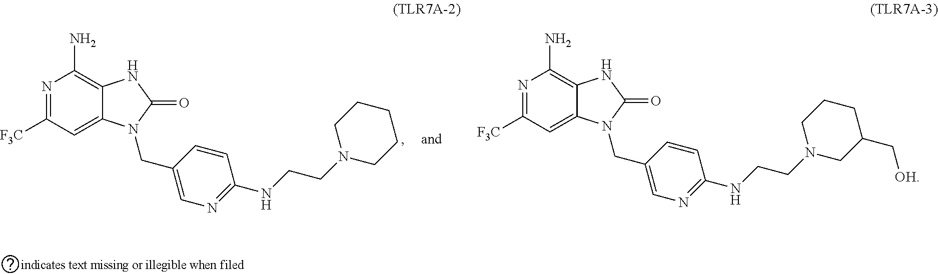 [SMILES: Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

Currently, there are no small molecules available for TLR9 and TLR3 agonists. Therefore, an oligonucleotide can be used for drug moiety in the combinations, compositions and methods hereof. Examples of TLR9 agonists include, but are not limited to, CpG-ODN (short, synthetic ssDNA containing unmethylated CpG dinucleotide motifs within particular sequence contexts), IMO-2055 (synthetic oligonucleotide containing unmethylated CpG dinucleotides), and 1018 ISS (short, synthetic unmethylated CpG oliodeoxynucleotide (CpG ODN)). A nonlimiting example of a TLR3 agonist includes poly (I:C) (polyinosine homopolymer annealed to a strand of polycytidine homopolymer).

In certain embodiments, any suitable immunomodulatory (e.g., immunoinhibitory) small molecule that binds to a PI3K can be used as the drug moiety. Non-limiting examples of PI3K antagonists include, but are not limited to:

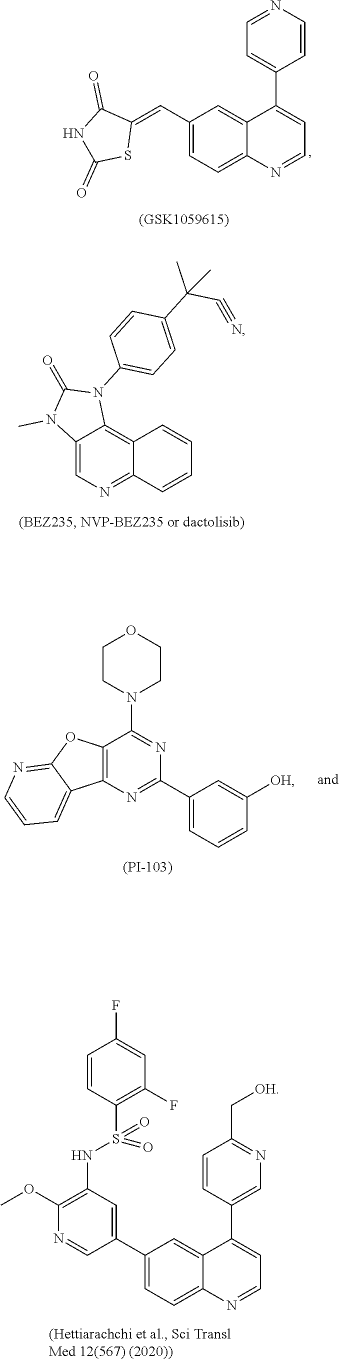 [SMILES: O=C1NC(=O)/C(=C/c2ccc3nccc(-c4ccncc4)c3c2)S1 ; Cn1c(=O)n(-c2ccc(C(C)(C)C#N)cc2)c2c3ccccc3ncc21 ; Oc1cccc(-c2nc(N3CCOCC3)c3oc4ncccc4c3n2)c1 ; COc1ncc(-c2ccc3nccc(-c4ccc(CO)nc4)c3c2)cc1NS(=O)(=O)c1ccc(F)cc1F]

Any suitable immunomodulatory (e.g., immunostimulatory) small molecule that binds to a STING can be used as the drug moiety. A non-limiting example of a STING agonist includes:

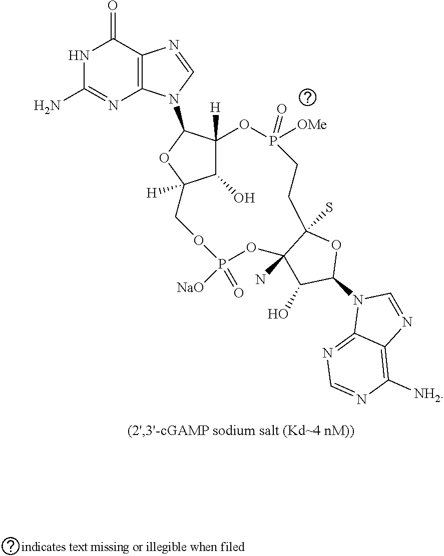

In certain embodiments, any suitable immunomodulatory (e.g., immunostimulatory) small molecule that binds to an NLR can be used for the drug moiety. A non-limiting example of an NLR agonist includes:

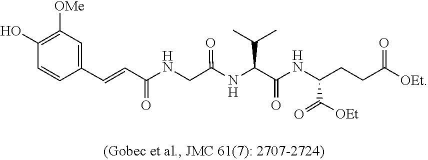 [SMILES: CCOC(=O)CC[C@@H](NC(=O)[C@@H](NC(=O)CNC(=O)/C=C/c1ccc(O)c(OC)c1)C(C)C)C(=O)OCC]

In some embodiments the SMDC is a folate-TLR7 agonist, a releasable form of a folate-(PEG)₃-TLR7 agonist, or a non-releasable form of a folate-(PEG)₃-TLR7 agonist.

It will be appreciated by those of skill in the art that compounds can exhibit polymorphism. Indeed, the compounds can comprise any racemic, optically-active, polymorphic, or stereoisomeric form, or mixtures thereof, of a compound described herein that exhibits the useful properties described, it being well-known in the art how to prepare optically active forms (for example, by resolution of the racemic form by recrystallization techniques, by synthesis from optically-active starting materials, by chiral synthesis, or by chromatographic separation using a chiral stationary phase) and how to determine antitumor activity using the standard tests described herein, or using other similar tests which are well-known in the art. Further, unless otherwise expressly stated, structures depicted herein are also meant to include all stereochemical forms of the structure, i.e., the right hand (R) and left hand (S) configurations of each asymmetric center. Therefore, single stereochemical isomers as well as enantiomeric and diastereomeric mixtures of the present compositions are within the scope of the present disclosure.

Specific values listed herein for radicals, substituents, and ranges are for illustration purposes only unless otherwise specified; such examples do not exclude other defined values or other values within defined ranges for the radicals and substituents. For example, (C₁-C₆)alkyl can be methyl, ethyl, propyl, isopropyl, butyl, iso-butyl, sec-butyl, pentyl, 3-pentyl, or hexyl; (C₁-C₃)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; (C₁-C₃)alkoxy can be methoxy, ethoxy, or propoxy; and (C₂-C₆)alkanoyloxy can be acetoxy, propanoyloxy, butanoyloxy, isobutanoyloxy, pentanoyloxy, or hexanoyloxy.

Further, where a moiety is substituted with an R substituent or a substituted group, the group may be referred to as “R-substituted.” Where a moiety is R-substituted or is otherwise described as generally comprising a substituted group, the moiety is substituted with at least one R substituent and each substituent is optionally different. It will be appreciated that the substituted group (or R substituent) may comprise any molecule or combination molecules provided the inclusion thereof does not substantially affect the overall structure and shape of the compound, nor alters any hydrogen bonds that are essential to the underlying compound achieving its intended purpose (e.g., binding to a targeted pattern recognition receptor).

Where substituent groups are specified by the conventional chemical formulae, written from left to right, they equally encompass the chemically identical substituents that would results from writing the structure from right to left, e.g., —CH₂O— is equivalent to —OCH₂—.

In certain embodiments, the drug (e.g., a TLR7 agonist) of the conjugates provided herein can be a radical having a structure of Formula XX or a functional fragment or analog thereof:

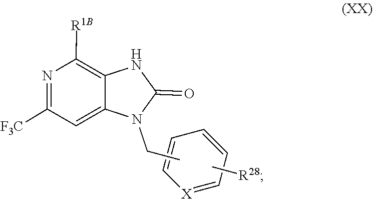

wherein,
R^{1B} is —NH₂ or —NH—R^{1X},
R^{2B} is a hydrogen (H), an alkyl, an alkenyl, an alkynyl, an alicyclic, an aryl, a biaryl, a heteroaryl, —NH—R^{2X}, —O—R^{2X}, —S—R^{2X},

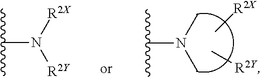

each of R^{1X}, R^{2X}, and R^{2Y} are independently selected from the group consisting of an H, an alkyl, an alkenyl, an alkynyl, an alicyclic, an aryl, a biaryl, and a heteroaryl,

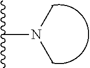 [SMILES: *N(C)C]

is a 3-10 membered nitrogen (N)-containing non-aromatic mono- or bicyclic heterocycle, and
X is CH or N.

In certain embodiments where the TLR7 agonist has Formula XX, the TLR7 agonist is conjugated to the targeting moiety at one of R^{1B}, X, or R^{2B} through a linker.

Alkyl, alkoxy, etc. denote a straight (i.e., unbranched) or branched chain, or a combination thereof, which may be fully saturated, mono- or polyunsaturated and can include di- and multivalent radicals, having the number of carbon (C) atoms designated (i.e., C₁-C₁₀ means one to ten carbons). Examples of saturated hydrocarbon radicals include, without limitation, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, without limitation, vinyl, 2-propenyl, crotyl-2-isopentenyl, 2-(butadienyl), 2,4-pentadienyl, 3-(1,4-penadienyl), ethynyl, 1- and 3-propynyl, 3-butynyl, and the higher homologs and isomers. An alkoxy is an alkyl attached to the remainder of the molecule via an oxygen linker (—O—). In some embodiments, alkoxy refers to a radical bonded through an oxygen atom of the formula —O-alkyl.

In general, the term “acyl” or “acyl substituent” refers to a derived by the removal of one or more hydroxyl groups from an oxoacid, including inorganic acids, and contains a double-bonded oxygen atom and an alkyl group. Further, reference to an individual radical such as “propyl” embraces only the straight chain radical, a branched chain isomer such as “isopropyl” being specifically referenced.

One of ordinary skill in the art will further appreciate that the above SMDCs can be “deuterated,” meaning one or more hydrogen atoms can be replaced with deuterium. As deuterium and hydrogen have nearly the same physical properties, deuterium substitution is the smallest structural change that can be made. Replacement of hydrogen with deuterium can increase stability in the presence of other drugs, thereby reducing unwanted drug-drug interactions, and can significantly lower the rate of metabolism (due to the kinetic isotope effect). By lowering the rate of metabolism, half-life can be increased, toxic metabolite formation can be reduced, and the dosage amount and/or frequency can be decreased.

In some embodiments, the TLR7 agonist has Formula XXa and the TLR7 agonist is conjugated to the targeting moiety at any one of R^{1A}, R^{2A}, R^{3A}, or R⁵ through a linker:

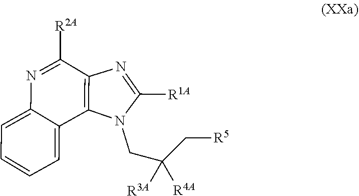

wherein:
R^{1A} is an optionally substituted C₃-C₈ alkyl (e.g., acyclic or cyclic);
R^{2A} is H, OH, NH₂, —SO₂NH₂, or N₃;
R^{3A} and R^{4A} are each independently alkyl, alkoxy, halogen, or cycloalkyl, wherein the alkyl, alkoxy, and cycloalkyl are optionally substituted; and
R⁵ is H, —OH, —NH₂, —SH, —SO₃H, —N₃, —COOH, —CONH₂, —SO₂NH₂.

### Ligand/Targeting Moiety

As noted above, the drug moiety of the SMDC is conjugated to a ligand (e.g., a targeting moiety). The ligand can comprise a folate receptor-binding ligand or a fibroblast activation protein (FAP) ligand. The ligand can be a “folate.” “Folate” refers to a folate receptor-binding molecule (e.g., FRβ), including for example folic acid and analogs and derivatives of folic acid such as, without limitation, folinic acid, pteroylpolyglutamic acid, pteroyl-D-glutamic acid, and folate receptor-binding pterdines such as tetrahydropterins, dihydrofolates, tetrahydrofolates, and their deaza and dideaza analogs.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs may include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate. Other folates useful as complex forming ligands in the context of the present disclosure are the folate receptor-binding analogs pemetrexed, proguanil, pyrimethamine, trimethoprim, pralatrexate, raltitrexed, aminopterin, amethopterin (also known as methotrexate), N¹⁰-methylfolate, 2-deamino-dydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

Folic acid and the foregoing analogs and/or derivatives are also termed “a folate,” “the folate,” or “folates” reflecting their ability to bind to folate-receptors. Such molecules, when conjugated with exogenous molecules, are effective to enhance transmembrane transport, such as via folate-mediated endocytosis. The foregoing can be used in the folate receptor-binding ligands described herein.

A pyrido[2,3-d]pyrimidine analog ligand (e.g., or radical thereof), a functional fragment or analog thereof, or any other molecule, fragment or atom with an affinity (for example, and without limitation, a high specificity) for FRβ can alternatively be used as the ligand (or radical thereof). For example, such folate analog molecules may have a relative affinity for binding FRβ of about 0.01 or greater as compared to folic acid at a physiological temperature of about 20° C./25° C./30° C. Similarly, a Galectin-3 ligand, a translocator protein (TSPO) ligand, and any other ligand or targeting moiety with a highly specific affinity for cancerous cells or tissue can be employed.

In some instances, FRβ is significantly upregulated in activated myeloid cells (e.g., predominantly activated monocytes and M2-like macrophages), for example, with all recorded data to date supporting that FRβ is only induced in cells of myelogenous origin following exposure to anti-inflammatory or proinflammatory stimuli. The folate receptor can be upregulated in (e.g., more than 90%) of non-mucinous ovarian carcinomas. In certain instances, the folate receptor is present in kidney, brain, lung, and breast carcinoma. For example, although there are a number of cancers that do not themselves express the folate receptor in sufficient numbers to provide the desired specificity, cancerous tumors do express myeloid-derived suppressor cells (MDSCs), for example, which do express FRβ and, for example, can be targeted by a ligand provided herein. In some embodiments, folate receptors are not substantially present (e.g., present only at extremely low levels) in healthy (non-myeloid) tissues (e.g., whether lungs, liver, spleen, heart, brain, muscle, intestines, pancreas, bladder, etc.). In some instances, even quiescent tissue-resident macrophages that are abundant throughout the body are predominantly FRβ-negative. In some instances, uptake of folate-targeted imaging agents is in, for example, inflamed tissues, malignant lesions, and the kidneys. In certain instances, subjects devoid of cancer only retain folate-targeted drugs in the kidneys and sites of inflammation. In some instances, the discrepancy in folate receptor expression provides a mechanism for selectively targeting fibrotic cancer cells.

In some embodiments, the conjugates/compounds and methods leverage the limited expression of FRβ to target/localize systemically administered potent compounds (e.g., conjugates or drugs) to fibrotic and/or cancerous tissue. In some instances, the compounds are delivered directly to FRβ expressing cells, for example, which advantageously prevents the systemic activation of the immune system and, for example, can avoid (e.g., at least a portion of) the toxicity that has heretofore prevented systemic use of non-targeting compounds (e.g., drugs). In some embodiments, the methods are used to treat cancers, for example, regardless of if the cancer expresses the folate receptor. In some embodiments, folic acid and other folate receptor-binding ligands (or radicals thereof), such as, for example folate, are used as ligands, since for example, they have affinity for FRβ.

Specific examples of suitable ligands (or radicals thereof) are set forth below; however, it will be understood that the ligand (or radical thereof) of the SMDC can comprise any ligand (or radical thereof) useful to target FRβ and is not limited to the structures specified herein. The ligand (or radical thereof) may bind to FRβ.

Compounds can include a ligand (or radical thereof) having a structure of formula V or a functional fragment or analog thereof:

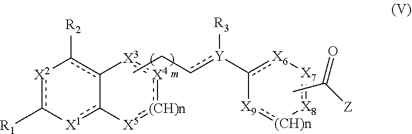

wherein
X₁, X₂, X₃, X₄, X₅, X₆, X₇, X₈, and X₉ are each independently nitrogen (N), NH, CH, CH₂, oxygen (O), or sulfur (S);
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

In a further aspect, by way of nonlimiting example, the ligand (or radical thereof) of formula V has a structure of VI (or a functional fragment or analog thereof):

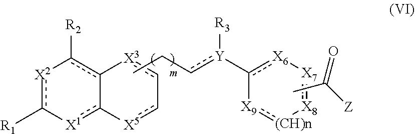

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

Another specific ligand (or radical thereof) of formula V (or a functional fragment or analog thereof) can have a structure of formula VII:

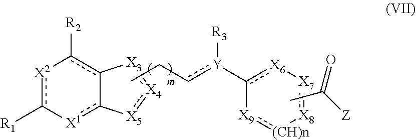

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m and n are each independently 0, 1, or between 0 and 1; and
[symbol] is representative of either a single or double bond C—C.

In certain embodiments, a ligand (or radical thereof) of formula VI can have the structure of formula VIII or a functional fragment or analog thereof:

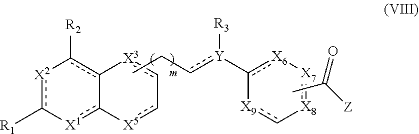

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C.

A ligand (or radical thereof) of formula VI can have the structure of formula IX (or a functional fragment or analog thereof):

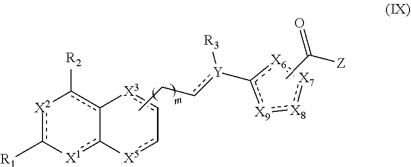

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C.

A ligand (or radical thereof) of formula VII can have the structure of formula X or XI (or a functional fragment or analog either):

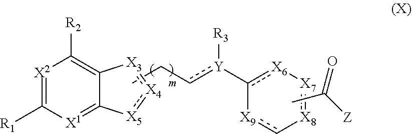

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and
[symbol] is representative of either a single or double bond C—C;
   or

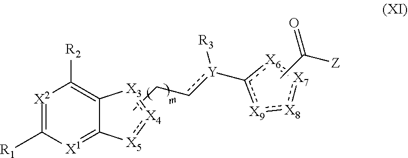

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or a substrate;
R₁ and R₂ are each independently NH₂, OH, SH, CH₃, or H;
R₃ is H or an alkyl;
m is 0, 1, or between 0 and 1; and [symbol] is representative of either a single or double bond C—C.

The chemical structures and spectroscopic data of additional ligands (e.g., or radicals thereof) are provided in Table 3, Table 4, Table 5 and Table 6.

Table 3 provides non-limiting examples of additional embodiments of a ligand (e.g., or radicals thereof) having the structure of formula VIII.

TABLE 3
Formula VIII

| Ligand | Structure |
|---|---|
| a | 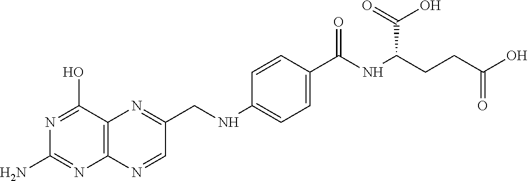 [SMILES: Nc1nc(O)c2nc(CNc3ccc(C(=O)N[C@@H](CCC(=O)O)C(=O)O)cc3)cnc2n1] |
| b | 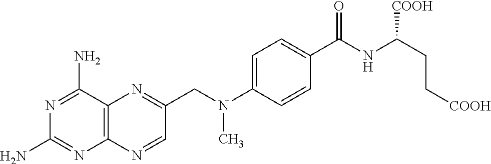 [SMILES: CN(Cc1cnc2nc(N)nc(N)c2n1)c1ccc(C(=O)N[C@@H](CCC(=O)O)C(=O)O)cc1] |
| c | 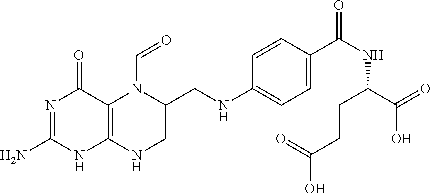 [SMILES: Nc1nc(=O)c2c([nH]1)NCC(CNc1ccc(C(=O)N[C@@H](CCC(=O)O)C(=O)O)cc1)N2C=O] |
| d | 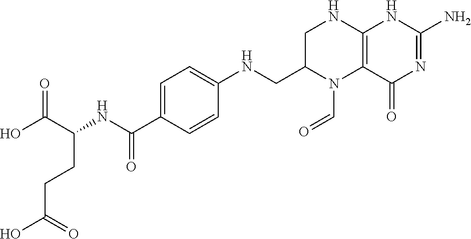 [SMILES: Nc1nc(=O)c2c([nH]1)NCC(CNc1ccc(C(=O)N[C@H](CCC(=O)O)C(=O)O)cc1)N2C=O] |
| e | 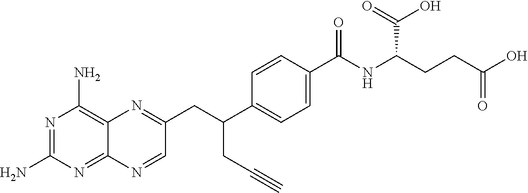 [SMILES: C#CCC(Cc1cnc2nc(N)nc(N)c2n1)c1ccc(C(=O)N[C@@H](CCC(=O)O)C(=O)O)cc1] |
| f | 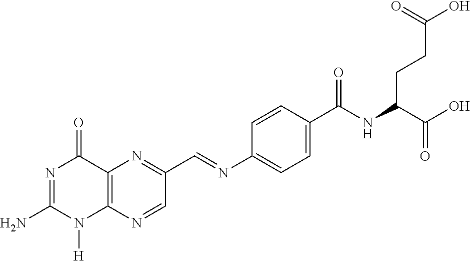 |
| g | 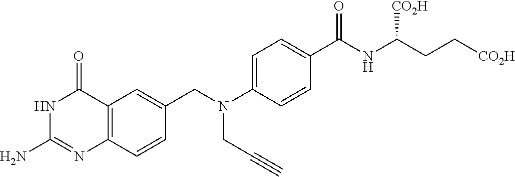 [SMILES: C#CCN(Cc1ccc2nc(N)[nH]c(=O)c2c1)c1ccc(C(=O)N[C@@H](CCC(=O)O)C(=O)O)cc1] |
| h | 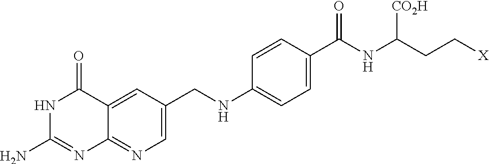 |
| | wherein X is PO(OH)₂, CH₂NH₂, or SO₂OH |
| i | 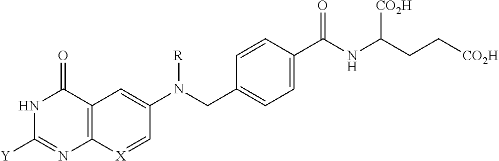 |
| | wherein |
| | X is N or CH; |
| | Y is NH₂, H, or CH₃; and |
| | R is H, CH₃, or CHO |
| j | 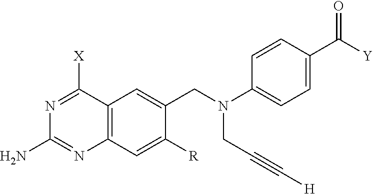 |
| | wherein |
| | X is OH or OCH₃; |
| | R is H or CH₃; and |
| | Y is glutamic acid, valine, or a substrate. |

Table 4 provides non-limiting examples of additional embodiments of a ligand (e.g., or radicals thereof) having the structure of formula IX.

TABLE 4
Formula IX

| Ligand | Structure |
|---|---|
| aa | 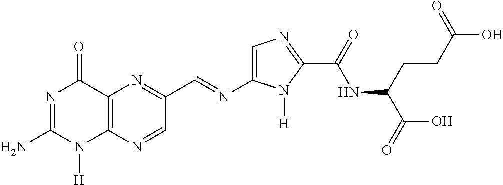 |
| bb | 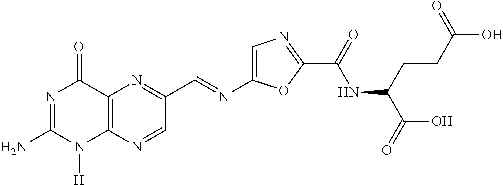 |
| cc | 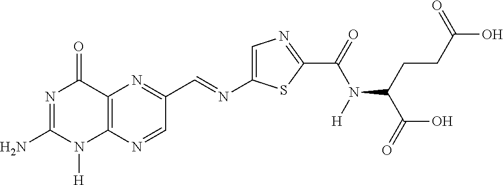 |
| dd | 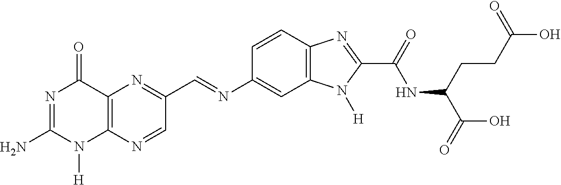 |
| ee | 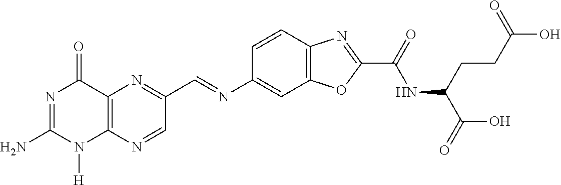 |
| ff | 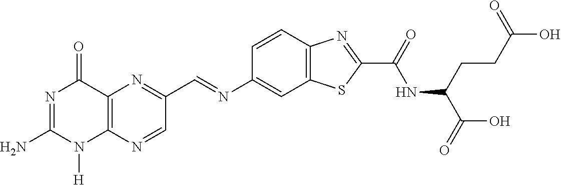 |

Table 5 provides non-limiting examples of additional embodiments of a ligand having the structure of formula X′.

TABLE 5
Formula X′

| Ligand | Structure |
|---|---|
| aaa | 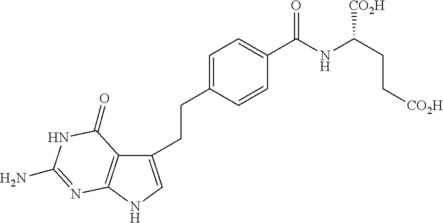 [SMILES: Nc1nc2[nH]cc(CCc3ccc(C(=O)N[C@@H](CCC(=O)O)C(=O)O)cc3)c2c(=O)[nH]1] |
| bbb | 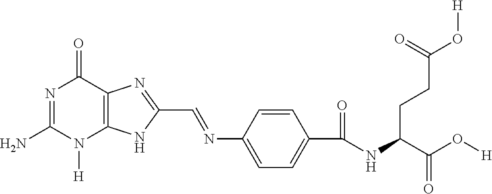 [SMILES: Nc1nc(=O)c2nc(/C=N/c3ccc(C(=O)N[C@@H](CCC(=O)O)C(=O)O)cc3)[nH]c2[nH]1] |
| ccc | 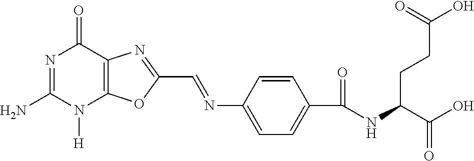 [SMILES: Nc1nc(=O)c2nc(/C=N/c3ccc(C(=O)N[C@@H](CCC(=O)O)C(=O)O)cc3)oc2[nH]1] |
| ddd | 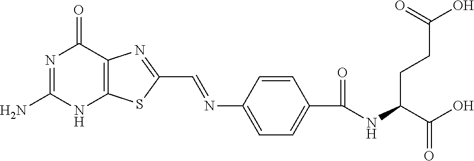 [SMILES: Nc1nc(=O)c2nc(/C=N/c3ccc(C(=O)N[C@@H](CCC(=O)O)C(=O)O)cc3)sc2[nH]1] |

As previously noted, instead of a folate, the ligand (e.g., a radical thereof) may be one or more nonclassical antifolate analogs such as, for example, pyrido[2,3-d]pyrimidine or similar analogs (or radicals thereof) having the formulae (e.g., radicals of the formulae) set forth in Table 6 below (or an analog or functional fragment thereof):

TABLE 6
Nonclassical antifolate analogs

| Ligand | Formula |
|---|---|
| aaaa | 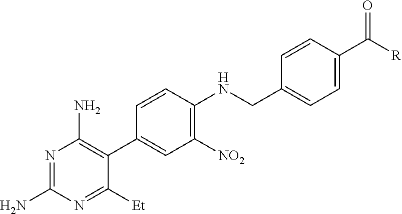 |
| | wherein R is NH₂, NHMe, NHCH(CO₂Et)(CH₂)₂CO₂Et, |
| | NHCH(CO₂Et)(CH₂)₂CO₂H, or |
| | 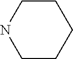 |
| bbbb | 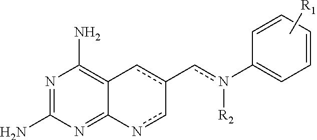 |
| | wherein |
| | R₁ is 3,4,5-(OCH₃)₃, 3,4-(OCH₃)₂, or 4-OCH₃; |
| | R₂ is an H, an alkyl chain or CHO; and |
| | [symbol] is representative of either a single or double bond C-C. |
| cccc | 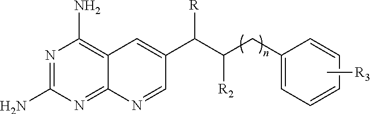 |
| | wherein |
| | n is 0 or 1; |
| | R₁ and R₂ are each independently an H or an alkyl; and |
| | R₃ is an H, 3′,4′,5′-Ome, 2′,3′,4′-Ome, 2′,4′,5′-Ome, |
| | 2′,4′,6′-Ome, 3′,4′-Ome, 3′,5′-Ome, 2′,5′-Ome, 2′,3′-C₄H₄, |
| | 4′-OMe,2′,3′-C₄H₄, 6′-OMe, 2′,3′-C₄H₄, 4′-O-C₆H₅ or 4′- |
| | CONH-L-glutamic acid. |
| dddd | 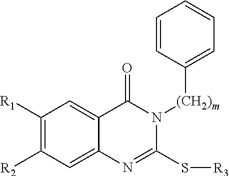 |
| | wherein |
| | n is 0 or 1; |
| | R₁ is CH₃, chloride (Cl) or OCH₃; |
| | R₂ is H or OCH₃; and |
| | R₃ is one of the following: |
| eeee | 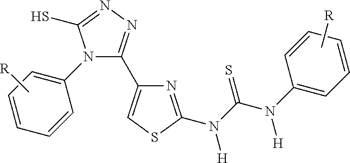 |
| | wherein R is H, 4-C1, 2-CH₃O, 4-CH₃O, 2,4-(CH₃O)₂, 4- |
| | CH₃, or 4-C₆H₅O. |
| ffff | 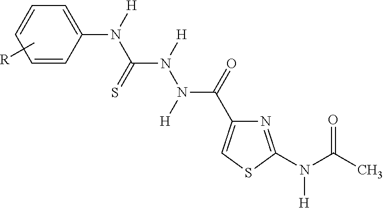 |
| | herein R is H, 4-Cl, 2-CH₃O, 4-CH₃O, 2,4-(CH₃O)2, 4- |
| | CH₃, or 4-C₆H₅O. |
| gggg | 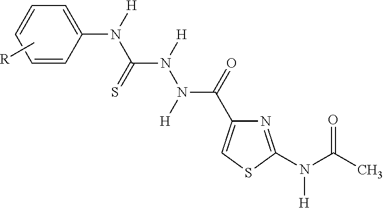 |
| | wherein R is H, 4-C1, 2-CH₃O, 4-CH₃O, 2,4-(CH₃O)₂, 4- |
| | CH₃, or 4-C₆H₅O. |
| hhhh | 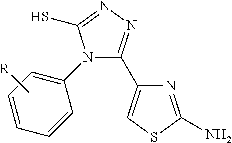 |
| | wherein R is H, 4-C1, 2-CH₃O, 4-CH₃O, 2,4-(CH₃O)₂, 4- |
| | CH₃, or 4-C₆H₅O. |
| iiii | 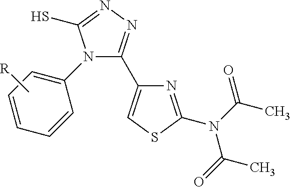 |
| | wherein R is H, 4-C1, 2-CH₃O, 4-CH₃O, 2,4-(CH₃O)2, 4- |
| | CH₃, or 4-C₆H₅O. |
| jjjj | 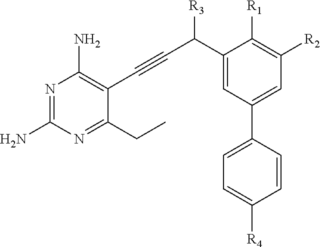 |
| | wherein |
| | R₁ and R₂ are each independently H or OMe; |
| | R₃ is H or an alkyl; and |
| | R₄ is o-COOH, m-COOH or p-COOH. |
| kkkk | 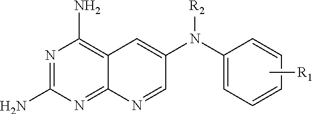 |
| | wherein |
| | R₁ is H, 2′-Ome, 4′-OMe or 2′,5′-diOMe, 3′,4′,5′-triOMe, |
| | 4′-Me, 4′-i-Pr, 3′,4′-(C₄H₄), 2′,3′-(C₄H₄), 4′-NO2, 2′,5′-diF |
| | or 3′,4′,5′--triF; and |
| | R₂ is H or an alkyl. |

### Linkers

In certain embodiments, the drug moiety and the ligand can be conjugated via a linker. As used herein, the term “linker” includes a chain of atoms that is bio-functionally adapted to form a chemical bond and connects a drug moiety and a ligand to form a conjugate. Illustratively, the chain of atoms can include carbon, nitrogen, oxygen, sulfur, silicon (Si), and phosphorus (P), such as C, N, O, S, and P, or C, N, O, and S. The linker can comprise a wide variety of links, such as in the range from about 2 to about 100 atoms in the contiguous backbone and can be releasable or non-releasable. The linker can comprise a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol. The linker can be (PEG)₃.

The term “releasable” in the context of a linker means a linker that includes at least one bond that can be broken (e.g., chemically or enzymatically hydrolyzed) under physiological conditions, such as, for example, by reducing agent-labile, pH-labile, acid-labile, base-labile, oxidatively labile, metabolically labile, biochemically labile, enzyme-labile or p-aminobenzylic based multivalent releasable bond. It is appreciated that the physiological conditions resulting in bond breaking do not necessarily include a biological or metabolic process and instead may include a standard chemical reaction, such as a hydrolysis reaction for example, at physiological pH or as a result of compartmentalization into a cellular organelle, such as an endosome having a lower pH than cytosolic pH. A cleavable bond can connect two adjacent atoms within the releasable linker and/or connect other linker portions or the targeting moiety/ligand and/or the drug, for example, at either or both ends of the releasable linker. In some instances, the releasable linker is broken into two or more fragments. In some instances, the releasable linker is separated from the targeting moiety/ligand. In some embodiments, the targeting moiety/ligand and the drug are released from each other and the drug becomes active.

In contrast, the term “non-releasable” in the context of a linker means a linker that includes at least one bond that is not easily or quickly broken under physiological conditions. A non-releasable linker can comprise a backbone that is stable under physiological conditions (e.g., the backbone is not susceptible to hydrolysis (e.g., aqueous hydrolysis or enzymatic hydrolysis)). A conjugate with a non-releasable linker does not release any component of the conjugate (e.g., a targeting ligand (e.g., a fully amorphous (FA)-ligand) or a drug (e.g., a TLR7 agonist)). The non-releasable linker can lack a disulfide bond (e.g., S—S) or an ester in the backbone. The conjugate can comprise a targeting moiety/ligand and a drug connected by a backbone that is substantially stable for the entire duration of the conjugate's circulation (e.g., during endocytosis into the target cell endosome). A conjugate comprising a non-releasable linker can be particularly beneficial when the drug targets TLRs, NOD-like receptors, and/or other pattern recognition receptors present within the endosome of a cell. Anon-releasable linker can comprise an amide, ester, ether, amine, and/or thioether (e.g., thio-maleimide). While specific examples are provided herein, it will be understood that any molecule(s) may be used in the non-releasable linker provided that at least one bond that is not easily or quickly broken under physiological conditions is formed.

Perhaps more specifically, a non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) of which will hydrolyze in an aqueous (e.g., buffered (e.g., phosphate buffer) solution) within a period of time (e.g., 24 hours). Where a non-releasable linker is employed, less than about ten percent (10%), and preferably less than five percent (5%) or none, of the administered conjugate can release the free drug (e.g., in systemic circulation prior to uptake by the targeted cells/tissue). For example, within one (1) hour of administration, less than five percent (5%) of the free drug can be released from the conjugate while the compound is in systemic circulation. This can be beneficial as it can reduce off-target toxicity of the free drug.

Both releasable and non-releasable linkers can be engineered to optimize biodistribution, bioavailability, andPK/PD (e.g., of the compound) and/or to increase uptake (e.g., of the conjugate or drug) into the targeted tissue pursuant to methodologies commonly known in the art or hereinafter developed, such as through PEGylation and the like. The linker can be configured to avoid significant release of a pharmaceutically active amount of the drug in circulation prior to capture by a cell (e.g., a cell of interest (e.g., a macrophage in a tissue to be treated)).

Conjugates comprising releasable linkers can be designed to diffuse across the membrane of the endosome and, for example, into the cytoplasm of a targeted cell. Releasable linkers can be designed such that the drug is not released until the conjugate reaches the cytoplasm.

A conjugate can comprise a releasable linker (e.g., to facilitate the release of the drug in the cytoplasm), for example where the drug comprises a PI3K kinase, IRAK, an activator of I_{K}β kinase (e.g., using prostratin or the like) or NF-_{K}β (see, e.g., Table 1), or a myeloid differentiation primary response 88 (MyD88) agonist. The releasable linker can prevent the release of the drug, for example, until after the targeting moiety binds the appropriate target (e.g., a macrophage folate receptor), is internalized into the endosome of the targeted cell, and/or diffuses into the cytoplasm (e.g., which is where the desired pattern recognition receptor is located). In certain embodiments, the releasable linker can release the drug within the endosome.

The drug (e.g., a TLR7 agonist) can be a radical having a structure of Formula XXX:

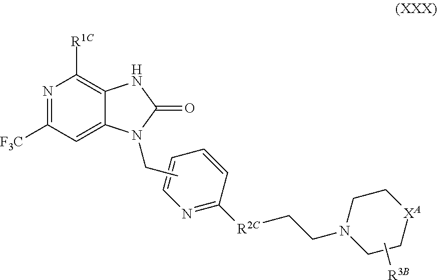

wherein,
R^{1C} is —NH₂ or —NH—R^{1X} or

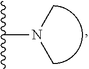 [SMILES: *N(C)C]

R^{2C} is a bond, NH, —NR^{1X}, or CH₂, R^{3B} is OH, H, NH₂, —CH₂OH, —CH₂—NH₂, —SO₂—NH₂, or COOH,
if applicable,

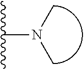 [SMILES: *N(C)C]

is a 3-10 membered N-containing non-aromatic mono- or bicyclic heterocycle;
X^{A} is CH₂, NH₂, or —NH—R^{1X}; and
each R^{1X} is independently selected from the group consisting of an H, an alkyl, an alkenyl, an alkynyl, an alicyclic, an aryl, a biaryl, and a heteroaryl, and
   where the drug is conjugated to the targeting moiety/ligand at one of R^{1C}, R^{2C}, or R^{3B} through a linker, such as “L” or “Lₙ”.

The linker Lₙ can be configured to avoid release of the compound and n can be an integer equal to or less than 50. In certain embodiments the linker Lₙ comprises a PEG linker or a PEG derivative linker, n is an integer selected from the range 1-32, and/or the targeting moiety/ligand is specific for FRβ. Thus, n can be 1-50, 1-32, 1-10, 2-8, or 2-4, for example.

In certain embodiments, L is a hydrolyzable linker. Alternatively, L can be a non-hydrolyzable linker. L also can be an optionally substituted heteroalkyl.

The term “alkylene,” by itself or as part of another substituent means, unless otherwise stated, a divalent radical derived from an alkyl, as exemplified, but not limited to, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combination(s) thereof, consisting of at least one carbon atom and at least one heteroatom selected from the group consisting of oxygen, nitrogen, phosphorous, silicone, and sulfur, and wherein the nitrogen and sulfur atoms may optionally be oxidized, and the nitrogen heteroatom may optionally be quartemized. The heteroatom(s) oxygen, nitrogen, phosphorous, sulfur, and silicone can be placed at any interior position of the heteroalkyl group or at the position at which the alkyl group is attached to the remainder of the molecule. Examples include, without limitation, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms can be consecutive, such as, for example, —CH₂—NH—OCH₃.

Similarly, the term “heteroalkylene,” by itself or as part of another substituent, means (unless otherwise stated) a divalent radical derived from heteroalkyl, as exemplified, but not limited by, —CH₂—CH₂—S—CH₂—CH₂ and —CH₂—S—CH₂—CH₂—NH—CH₂. For heteroalkylene groups, heteroatoms can also occupy either terminus or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

L can be a substituted heteroalkyl comprising at least one substituent selected from the group consisting of alkyl, hydroxyl, oxo, PEG, carboxylate, and halo. “Halo” or “halogen,” by itself or as part of another substituent, means, unless otherwise stated, a fluorine, chlorine, bromine, or iodine atom.

In certain embodiments, L can comprise a spacer (e.g., as described elsewhere herein). The spacer can comprise a peptidoglycan or a sugar, for example.

In certain embodiments, L can be a substituted heteroalkyl with at least one disulfide bond in the backbone thereof. In certain embodiments, L can be a peptide with at least one disulfide bond in the backbone thereof.

The terms “polypeptide,” “peptide,” and “protein” are used interchangeably herein to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

In certain embodiments, L can comprise —CONH—CH(COOH)—CH₂—S—S—CH₂—CRₐR_{b}—O—CO—, —CONH—CH(COOH)CRₐR_{b}—O—CO—, —C(O)NHCH(COOH)(CH₂)₂—CONH—CH(COOH)CRₐR_{b}—O—CO— or —C(O)NHCH(COOH)(CH₂)₂—CONH—CH(COOH)—CH₂—S—S—CH₂—CRₐR_{b}—O—CO—, wherein Rₐ and R_{b} are independently an H, an alkyl, or a heteroalkyl (e.g., PEG).

In certain embodiments, L can comprise a structure of:

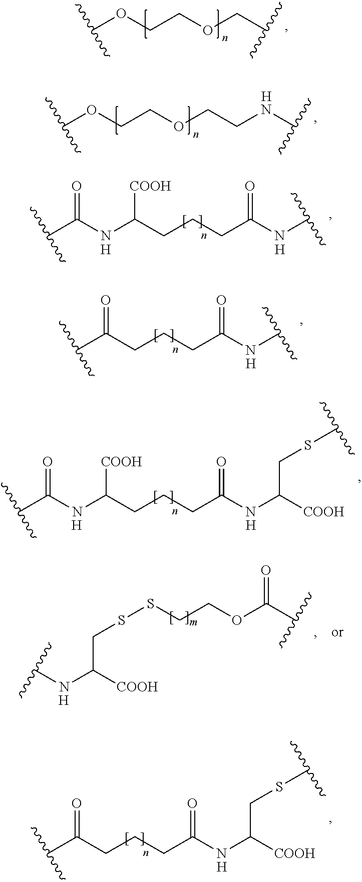

wherein n or m (where applicable) is 0 to 10.

In certain embodiments, L can comprise a structure of:

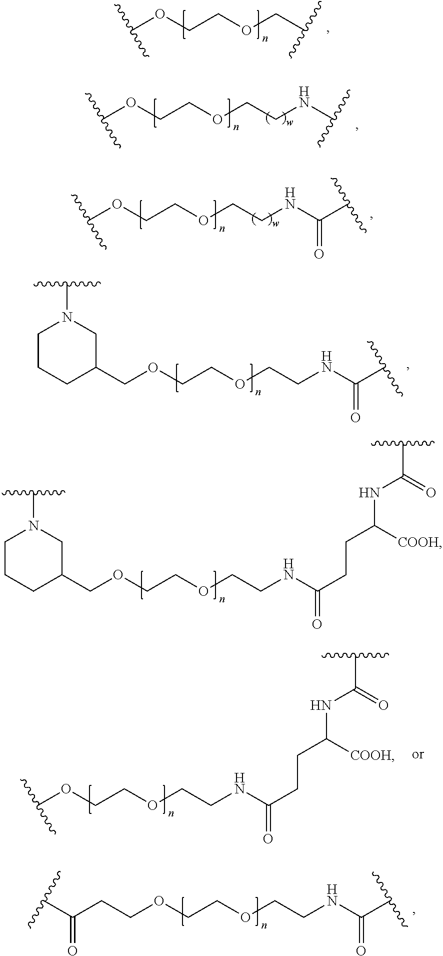

wherein n is 1 to 32. In at least one examplary embodiment, n is 1 to 30 and w is 0 to 5 (where applicable).

In certain embodiments, L can comprise the structure of:

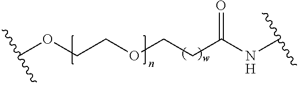

wherein n is 1 to 30 and w is 0 to 5.

The conjugate can comprise a ligand comprising a folate ligand or a functional fragment or analog thereof attached to a drug comprising a TLR agonist via a linker, wherein the TLR agonist has a structure represented by formula XXX′ or a pharmaceutically acceptable salt thereof:

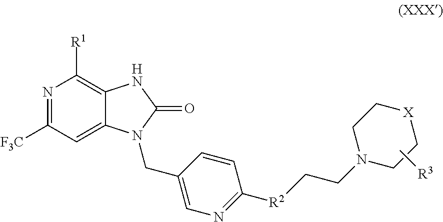

wherein:
R¹ is an amine group,
R² is a single bond —NH—,
R³ is an H, an alkyl, a hydroxy group, or any other substituted group thereof,
X is a CH₂, NH, O, or S, and
the linker is attached at R¹, R² or R³.

Additionally or alternatively, R₁ can be —NH₂ or —NH—R_{1X}; R₂ can be an H, an alkyl, an alkenyl, an alkynyl, an alicyclic, an aryl, a biaryl, a heteroaryl, —NH—R_{2X}, —O—R_{2X}x, —S—R_{2X},

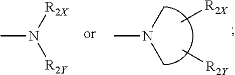

with each of R_{1X}, R_{2X}, and R_{2Y} independently selected from the group consisting of an H, an alkyl, an alkenyl, an alkynyl, an alicyclic, an aryl, a biaryl, and a heteroaryl;

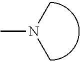

is a 3-10 membered N-containing non-aromatic mono- or bicyclic heterocycle; and/or X is CH, CR₂, or N.

When the conjugate comprises a TLR agonist (e.g., a radical thereof or a pharmaceutically acceptable salt thereof), for example and without limitation, a TLR3 agonist, a TLR7 agonist, a TLR7/8 agonist, a TLR8 agonist, or a TLR9 agonist (e.g., all of which bind with a TLR present within the endosome of a cell), the TLR agonist can be selected from the compounds listed in Table 2 or a pharmaceutically acceptable salt thereof.

TABLE 2
TLR agonists.

| Compound | Structure/Description | Type |
|---|---|---|
| A | 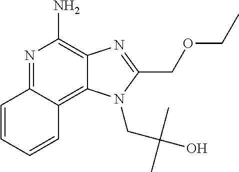 [SMILES: CCOCc1nc2c(N)nc3ccccc3c2n1CC(C)(C)O] | TLR7 agonist |
| B | 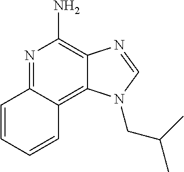 [SMILES: CC(C)Cn1cnc2c(N)nc3ccccc3c21] | TLR7 agonist |
| C | 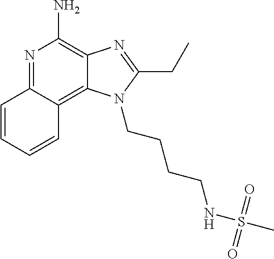 [SMILES: CCc1nc2c(N)nc3ccccc3c2n1CCCCNS(C)(=O)=O] | TLR7 agonist |
| D | 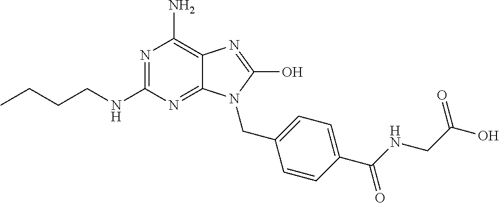 [SMILES: CCCCNc1nc(N)c2nc(O)n(Cc3ccc(C(=O)NCC(=O)O)cc3)c2n1] | TLR7 agonist |
| E | 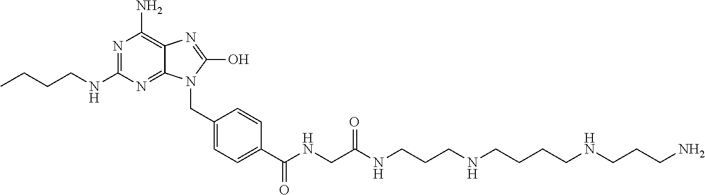 [SMILES: CCCCNc1nc(N)c2nc(O)n(Cc3ccc(C(=O)NCC(=O)NCCCNCCCCNCCCN)cc3)c2n1] | TLR7 agonist |
| F | 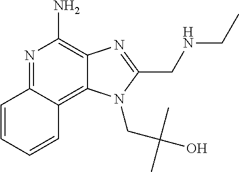 [SMILES: CCNCc1nc2c(N)nc3ccccc3c2n1CC(C)(C)O] | TLR7 agonist |
| G | 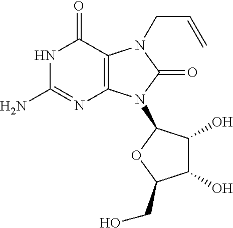 [SMILES: C=CCn1c(=O)n([C@@H]2O[C@H](CO)[C@@H](O)[C@H]2O)c2nc(N)[nH]c(=O)c21] | TLR7 agonist |
| H | 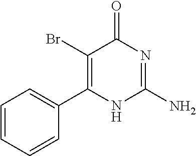 [SMILES: Nc1nc(=O)c(Br)c(-c2ccccc2)[nH]1] | TLR7 agonist |
| I | 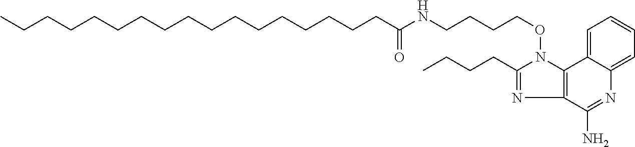 [SMILES: CCCCCCCCCCCCCCCCCC(=O)NCCCCOn1c(CCCC)nc2c(N)nc3ccccc3c21] | TLR7 agonist |
| J | 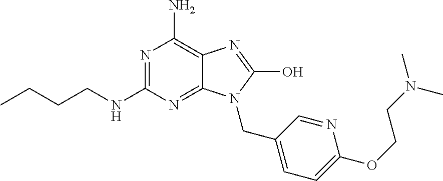 [SMILES: CCCCNc1nc(N)c2nc(O)n(Cc3ccc(OCCN(C)C)nc3)c2n1] | TLR7 agonist |
| K | 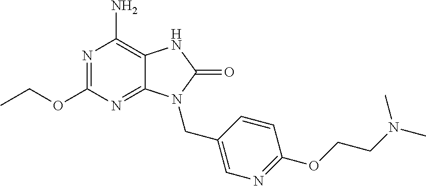 [SMILES: CCOc1nc(N)c2[nH]c(=O)n(Cc3ccc(OCCN(C)C)nc3)c2n1] | TLR7 agonist |
| L | 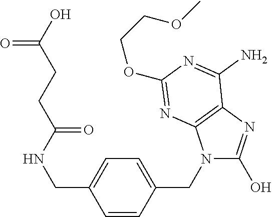 [SMILES: COCCOc1nc(N)c2nc(O)n(Cc3ccc(CNC(=O)CCC(=O)O)cc3)c2n1] | TLR7 agonist |
| M | 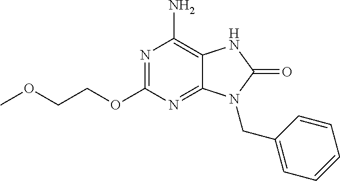 [SMILES: COCCOc1nc(N)c2[nH]c(=O)n(Cc3ccccc3)c2n1] | TLR7 agonist |
| N | 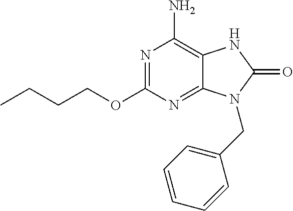 [SMILES: CCCCOc1nc(N)c2[nH]c(=O)n(Cc3ccccc3)c2n1] | TLR7 agonist |
| O | 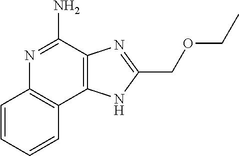 [SMILES: CCOCc1nc2c(N)nc3ccccc3c2[nH]1] | TLR7/8 agonist |
| P | 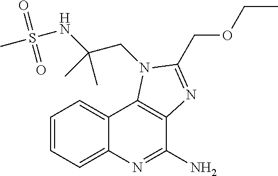 [SMILES: CCOCc1nc2c(N)nc3ccccc3c2n1CC(C)(C)NS(C)(=O)=O] | TLR7/8 agonist |
| Q | See, e.g., Lipanov et al., ″The structure of poly(dA): poly(dT) in a | TLR7/8 |
| | condensed state and in solution,″ Nucleic Acids Research 15 (14): | agonist |
| | 5833-5844 (1987). | |
| R | 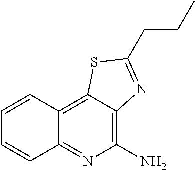 | TLR8 agonist |
| S | 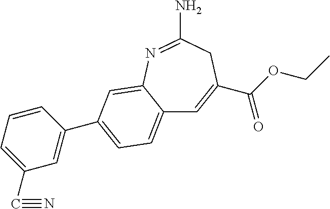 [SMILES: CCOC(=O)C1=Cc2ccc(-c3cccc(C#N)c3)cc2N=C(N)C1] | TLR8 agonist |
| T | 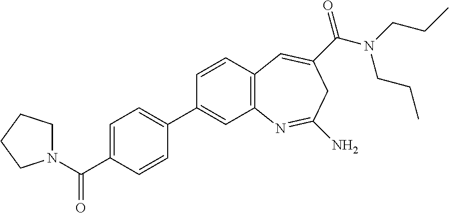 [SMILES: CCCN(CCC)C(=O)C1=Cc2ccc(-c3ccc(C(=O)N4CCCC4)cc3)cc2N=C(N)C1] | TLR8 agonist |
| U | Short synthetic single-stranded DNA molecules containing | TLR9 |
| | unmethylated CpG dinucleotides in particular sequence contexts | agonist |
| | (CpG motifs) (CpG ODN) | |
| V | Synthetic oligonucleotide containing unmethylated CpG | TLR9 |
| | dinucleotides with potential immuno-potentiating activity (IMO | agonist |
| | 2005) | |
| W | Short, synthetic, unmethylated CpG oligodeoxynucleotide (CpG | TLR9 |
| | ODN) with immunostimulatory activity (1018-ISS) | agonist |
| X | Comprises a strand of inosine poly(I) homopolymer annealed to a | TLR3 |
| | strand of cytidine (poly(I:C)) | agonist |
| Y | Poly(C)homopolymer | TLR3 |
| | | agonist |
| Z | 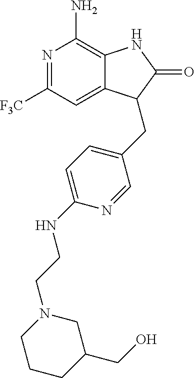 [SMILES: Nc1nc(C(F)(F)F)cc2c1NC(=O)C2Cc1ccc(NCCN2CCCC(CO)C2)nc1] | TLR7 agonist |

In certain embodiments, the conjugate can comprise a structure of formula I:

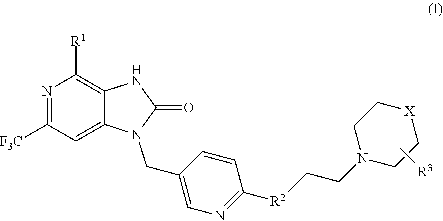

or a pharmaceutically acceptable salt thereof. R¹ can be an amine. R² can be a (e.g., single) bond, or an amine (e.g., —NH—). R³ can be an H, an alkyl, a hydroxy group, or any other suitable substituent. X can be CH₂, NH, O, or S. When the conjugate comprises a radical of formula I, a targeting moiety/ligand can be conjugated or connected thereto at any suitable location, such as at or through R¹, R², and/or R³ (e.g., through a linker and/or directly).

For example, in certain embodiments, a conjugate can comprise a structure (or radical) of formula Ia:

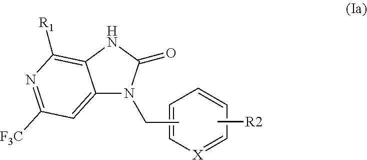

or a pharmaceutically acceptable salt thereof. X can be a CH or an N. R₁ can be —NH₂ or —NH—R_{1X}. R₂ can be H, alkyl, alkenyl, alkynyl, alicyclic, aryl, biaryl, heteroaryl, —NH—R_{2X}, —O—R_{2X}, —S—R_{2X},

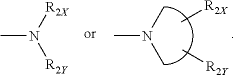

Each of R_{1X}, R_{2X}, and R_{2Y} can be independently selected from the group consisting of H, alkyl, alkenyl, alkynyl, alicyclic, aryl, biaryl, and heteroaryl.

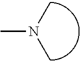

can be a 3-10 membered N-containing non-aromatic mono- or bicyclic heterocycle. When a conjugate comprises a radical of formula Ia, a targeting moiety/ligand can be conjugated or connected thereto at any suitable location, such as at or through R¹, R², and/or R³ (e.g., through a linker and/or directly).

A conjugate can comprise a structure (or radical) of formula II:

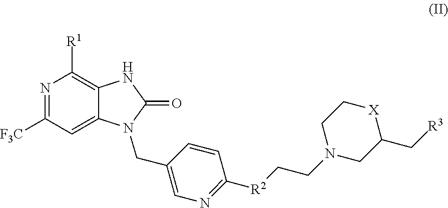

or a pharmaceutically acceptable salt thereof. In certain embodiments, R¹ can be an amine. In certain embodiments, R² can be a (e.g., single) bond or —NH—. In certain embodiments, R³ can be an H, an alkyl, an hydroxy group, or any other substituent. In certain embodiments, X can be a CH₂, NH, O, or S. When a conjugate comprises a radical of formula II, a targeting moiety/ligand can be conjugated or connected thereto at any suitable location, such as at or through R¹, R², and/or R³ (e.g., through a linker and/or directly).

In certain embodiments, the conjugate comprises a structure of formula III or a pharmaceutically acceptable salt thereof:

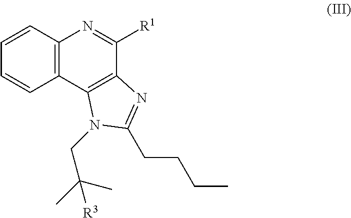

wherein, R¹ is an amine group and R³ is a hydroxy group. A targeting moiety (e.g., or a radical thereof) or other ligand can be conjugated at R¹ or R³ (through a linker or directly).

In certain embodiments, the conjugate comprises a structure (e.g., or radical thereof) of formula IV or a pharmaceutically acceptable salt thereof:

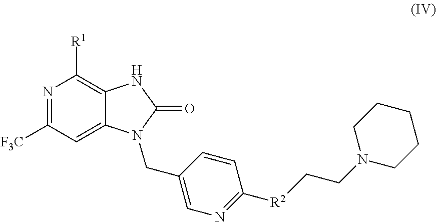

wherein R¹ is an amine group and R² is a single bond —NH—.

### Compounds and Administration

More than one SMDC can be administered and, in some instances, the SMDCs can comprise different drugs. For example, the different drugs of each SMDC can be selected from a TLR7 agonist and a TLR9 agonist. One or more SMDCs can be administered in a composition along with one or more conjugated and/or unconjugated drugs. The SMDCs and drugs described herein can be used in accordance with the methods described herein and, in some instances, depending on the desired application, can be combined with other drugs that deplete or inhibit myeloid-derived suppressor cells (e.g., in connection with treatment for cancer), and/or other anticancer drugs and therapies.

The compounds comprising the at least one SMDC can be prepared by conventional methods of organic synthesis practiced by those skilled in the art. The general reaction sequences outlined below represent a general method useful for preparing the compounds and are not meant to be limiting in scope or utility.

Descriptions of compounds are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art, thereby avoiding inherently unstable compounds.

A pharmaceutical composition is also provided. As used herein, the term “composition” generally refers to any product comprising more than one ingredient, e.g., one or more conjugates such as SMDCs. It is to be understood that the compositions described herein can be prepared from isolated conjugates or from salts, solutions, hydrates, solvates, and other forms. It is appreciated that certain functional groups, such as the hydroxy, amino, and like groups can form complexes with water and/or various solvents, in the various physical forms of the conjugates. It is also to be understood that the compositions can be prepared from various amorphous, non-amorphous, partially crystalline, crystalline, and/or other morphological forms of the conjugates, and the compositions can be prepared from various hydrates and/or solvates of the conjugates. Accordingly, pharmaceutical compositions that recite the conjugates described herein include each of, or any combination of, or individual forms of, the various morphological forms and/or solvate or hydrate forms of the conjugates described herein.

In certain embodiments, the pharmaceutical composition comprises a conjugate as described herein, such as at least one SMDC comprising a drug moiety conjugated to a ligand (e.g., a targeting moiety) via a linker, such as PEG or a derivative thereof.

Conjugates described herein can be administered in unit dosage forms and/or compositions containing one or more pharmaceutically acceptable carriers, adjuvants, diluents, excipients, and/or vehicles, and combinations thereof. The term “administering” and its formatives generally refer to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. Similarly, “pharmaceutically acceptable salt” refers to those salts with counter ions which may be used in pharmaceuticals. Such salts can include, without limitation: (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include arginine, benzathine, calcium, choline, diethylamine, diolamine, glycine, lysine, magnesium, meglumine, olamine, potassium, sodium, tromethamine and zinc salts. Hemi-salts of acids and bases also can be formed, for example, hemisulphate and hemicalcium salts.

The conjugates can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrastemal, intracranial, intratumoral, intramuscular, topical, inhalation and/or subcutaneous routes. Indeed, a conjugate, or composition comprising the same, can be administered directly into the blood stream, into muscle, or into an internal organ.

For example, the conjugate can be systemically administered (orally, for example) in combination with a pharmaceutically acceptable vehicle, such as an inert diluent or an assimilable edible carrier. For oral therapeutic administration, the conjugate can be combined with one or more excipients and used in the form of ingestible tablets, buccal tablets, troches, capsules, elixirs, suspensions, syrups, wafers, and the like. The percentage of the compositions and preparations may vary and may be between about 1 to about 99% weight of the active ingredient(s) and a binder, excipients, a disintegrating agent, a lubricant, and/or a sweetening agent (as are known in the art). The amount of active conjugate (e.g., at least one SMDC or drug thereof) in such therapeutically useful compositions is such that an effective dosage level will be obtained.

The preparation of parenteral conjugates/compositions under sterile conditions, for example, by lyophilization, can readily be accomplished using standard pharmaceutical techniques well known to those skilled in the art. The solubility of a conjugate used in the preparation of a parenteral composition may be increased by the use of appropriate formulation techniques, such as the incorporation of solubility-enhancing agents.

As previously noted, the conjugates/compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9), but, for some applications, they can be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations may further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents such as parabens, chlorobutanol, phenol, sorbic acid, thimerosal, and the like. In certain cases, it will be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the conjugate in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

For topical administration, it can be desirable to administer a conjugate to the skin as a composition or a formulation in combination with a dermatologically acceptable carrier, which may be a solid or a liquid. For example, in certain embodiments, solid carriers can include finely divided solids such as talc, clay, microcrystalline cellulose, silica, alumina and the like. Similarly, useful liquid carriers can comprise water, alcohols or glycols or water-alcohol/glycol blends, in which the conjugate can be dissolved or dispersed at effective levels, optionally with the aid of non-toxic surfactants. Additionally or alternatively, adjuvants, such as fragrances and antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

### CAR-Expressing Cytotoxic Lymphocyte Compositions

As previously noted, in addition to the at least one SMDC, the combinations and methods can include engineered CAR-expressing cytotoxic lymphocyte compositions. The cytotoxic lymphocytes can be cytotoxic T cells, natural killer (NK) cells, lymphokine-activated killer (LAK) cells, or a combination of two or more of the foregoing. In at least one embodiment, T lymphocytes (e.g., cytotoxic T lymphocytes) are engineered to express CAR.

The CAR is a fusion protein comprising a recognition region, a co-stimulation domain, and an activation signaling domain. In certain embodiments, the CAR binds a cell-surface antigen on an immunosuppressive cell or a cancerous cell with high specificity.

In certain embodiments, the recognition region of the CAR can be a scFv of an antibody, a Fab fragment or the like that binds to a cell-surface antigen (e.g., cluster of differentiation 19 (CD19)) with specificity (e.g., high specificity). Where the recognition region of the CAR comprises a scFv region, the scFv region can be prepared from (i) an antibody known in the art that binds a targeting moiety, (ii) an antibody newly prepared using at least one targeting moiety such as a hapten, and (iii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

“Percent (%) sequence identity” with respect to a reference to a polypeptide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

The co-stimulation domain of a CAR can serve to enhance the proliferation and survival of the cytotoxic lymphocytes upon binding of the CAR to a targeting moiety. In certain embodiments, the co-stimulation domain of the CAR can be CD28 (cluster of differentiation 28), CD137 (cluster of differentiation 137; 4-1BB), CD134 (cluster of differentiation 134; OX40), CD278 (cluster of differentiation 278; ICOS), CD2 (cluster ofdifferentiation 2), CD27 (cluster of differentiation 27), CD40L (cluster of differentiation 2; CD154), DAP10, NKG2D, signaling lymphocytic activation molecule (SLAM)-related receptor family (such as 2B4), TLRs or combinations thereof. A skilled artisan will understand that sequence variants of these co-stimulation domains can be used without adversely impacting the invention, where the variants have the same or similar activity as the domain upon which they are modeled. In various embodiments, such variants can have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the domain from which they are derived.

In certain embodiments, the activation signaling domain generates a lymphocyte activation signal upon binding of the CAR to a targeting moiety. Suitable activation signaling domains can be, without limitation, a T cell CD3ζ chain, a CD3ζ delta receptor protein, mbl receptor protein, B29 receptor protein or a Fc receptor γ. The skilled artisan will understand that sequence variants of these activation signaling domains can be used where the variants have the same or similar activity as the domain upon which they are modeled. In various embodiments, the variants have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the domain from which they are derived.

In an exemplary embodiment, the recognition region is a scFv region of an anti-FITC (fluorescein isothiocyanate) antibody, the co-stimulation domain is CD28, and the activation signaling domain is a T cell CD3ζ chain. The recognition region can be a scFv region of an anti-CD19 antibody, the co-stimulation domain can be CD137 (4-lBB), and the activation signaling domain can be a T cell CD3ζ chain.

In certain exemplary embodiments, the recognition region is a scFv region of an anti-CD19 antibody, the co-stimulation domain is CD28, and the activation signaling domain is a T cell CD3ζ chain.

Constructs encoding the CARs can be prepared using genetic engineering techniques. Such techniques are described in detail in Sambrook et al., “Molecular Cloning: A Laboratory Manual,” 3rd Edition, Cold Spring Harbor Laboratory Press, (2001), and Green and Sambrook, “Molecular Cloning: A Laboratory Manual,” 4th Edition, Cold Spring Harbor Laboratory Press, (2012), which are both incorporated herein by reference in their entireties (collectively, the “Protocols”).

By way of non-limiting examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction.

Other arrangements are also acceptable and include a recognition region, an activation signaling domain, and one or more co-stimulation domains.

The term “vector” means any nucleic acid that functions to carry, harbor, or express a nucleic acid of interest. Nucleic acid vectors can have specialized functions such as expression, packaging, pseudotyping, or transduction. Vectors can also have manipulatory functions if adapted for use as a cloning or shuttle vector. The structure of the vector can include any desired form that is feasible to make and desirable for a particular use. Such for can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized.

The placement of the recognition region in the fusion protein will generally be such that display of the region on the exterior of the cell is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3ζ transmembrane domain), and ahinge domain (e.g., CD8α hinge) that imparts flexibility to the recognition region and allows strong binding to the targeting moiety.

In an examplary embodiment, the CAR has a recognition region that is a scFv region of an anti-FITC antibody, a co-stimulation domain that is CD28, and an activation signaling domain that is a T cell CD3ζ chain. It is well-known to the skilled artisan that an anti-FITC scFv and an anti-fluorescein scFv are equivalent terms.

Cytotoxic lymphocytes (e.g., cytotoxic T lymphocytes) can be genetically engineered to express CAR constructs by transfecting a population of the lymphocytes with an expression vector encoding the CAR construct. Suitable methods for preparing a transduced population of lymphocytes expressing a selected CAR construct are well-known to the skilled artisan.

In one embodiment, the lymphocytes (e.g., cytotoxic T lymphocytes used to prepare CAR-T cells) used in the combinations, compositions, and methods described herein, can be autologous cells, although heterologous cells can also be used, such as when the patient being treated has received high-dose chemotherapy or radiation treatment to destroy the patient's immune system. In one embodiment, allogenic cells can be used.

The lymphocytes can be obtained from a subject by means well-known in the art. For example, T cells (e.g., cytotoxic T cells) can be obtained by collecting peripheral blood from the subject, subjecting the blood to Ficoll density gradient centrifugation, and then using a negative T cell isolation kit (such as EasySep™ T Cell Isolation Kit) to isolate a population of T cells from the peripheral blood.

In certain embodiments, the population of lymphocytes (e.g., cytotoxic T cells) need not be pure and may contain multiple types of cells, such as T cells, monocytes, macrophages, NK cells, and B cells. Further, in at least one embodiment, the population being collected can comprise at least about 90% of the selected cell type, at least about 91%, 92%, 93%, 94%, 95%, 96%, 97%, 98%, 99%, or 100% of the selected cell type.

Generally, after the lymphocytes are obtained, the cells are cultured under conditions that promote the activation of the cells. In at least one embodiment, the culture conditions are such that the cells can be administered to a subject without concern for reactivity against components of the culture medium. For example, the culture conditions may not include bovine serum products, such as bovine serum albumin. In one aspect, the activation can be achieved by introducing known activators into the culture medium, such as anti-CD3 antibodies in the case of cytotoxic T cells. Other suitable activators are generally known and include, for example, anti-CD28 antibodies. The population of lymphocytes can be cultured under conditions promoting activation for about 1 to about 4 days, for example. The appropriate level of activation can be determined by cell size, proliferation rate, or activation markers determined by flow cytometry.

In at least one embodiment, after the population of lymphocytes has been cultured under conditions promoting activation, the cells are transfected with an expression vector encoding a CAR. Suitable vectors and transfection methods for use in various embodiments are described above. After transfection, the cells can be immediately administered to the patient or the cells can be cultured for a time period to allow time for the cells to recover from the transfection, for example, at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days. In one aspect, suitable culture conditions can be similar to the conditions under which the cells were cultured for activation either with or without the agent that was used to promote activation.

Thus, as described above, the methods of treatment described herein can further comprise 1) obtaining a population of autologous or heterologous cytotoxic lymphocytes (e.g., cytotoxic T lymphocytes used to prepare CAR-T cells), 2) culturing the cytotoxic lymphocytes under conditions that promote the activation of the cells, and 3) transfecting the cytotoxic lymphocytes with an expression vector encoding a CAR to form CAR-expressing cytotoxic lymphocytes.

When the cells have been transfected and activated, a composition comprising the CAR-expressing cytotoxic lymphocytes can be prepared and administered to the subject. In at least one embodiment, culture media that lacks any animal products, such as bovine serum, can be used to culture the CAR-expressing cytotoxic lymphocytes. In another embodiment, tissue culture conditions typically used by the skilled artisan to avoid contamination with bacteria, fungi and mycoplasma can be used. In certain embodiments, prior to being administered to a patient, the cells (e.g., CAR-T cells) are pelleted, washed, and are resuspended in a pharmaceutically acceptable carrier or diluent.

Examplary compositions comprising CAR-expressing cytotoxic lymphocytes include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. In certain embodiments, depending on the identity of the culture medium, the CAR-expressing cytotoxic lymphocytes can be administered in the culture media as the composition, or concentrated and resuspended in the culture medium before administration. In various embodiments, the CAR-expressing cytotoxic lymphocyte composition can be administered to the subject via any suitable means, such as parenteral administration, e.g., intradermally, subcutaneously, intramuscularly, intraperitoneally, intravenously, or intrathecally.

In one aspect, the total number of CAR-expressing cytotoxic lymphocytes and the concentration of the cells in the composition administered to the patient will vary depending on a number of factors including the type of lymphocytes (e.g., cytotoxic T lymphocytes) being used, the binding specificity of the CAR, the identity of the cancer, the location of the cancer in the patient, the means used to administer the compositions to the patient, and the health, age and weight of the patient being treated. In various embodiments, suitable compositions comprising transduced CAR-expressing cytotoxic lymphocytes include those having a volume of about 0.1 ml to about 200 ml and about 0.1 ml to about 125 ml.

### Vector Compositions

Certain embodiments of the compositions and methods can include vector compositions. In some embodiments, the composition comprises a vector comprising a promoter operatively linked to a nucleic acid sequence encoding a CAR construct described herein. In some embodiments, the vector composition comprises lentiviral particles that carry a nucleic acid sequence encoding a CAR described herein. In some embodiments, the vector composition comprises a therapeutically effective amount of such lentiviral particles.

A lentivirus is a non-limiting example of a vector system that can be used. Lentiviruses are complex retroviruses that, in addition to the common retroviral genes Gag, Pol and Env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Lentiviral vectors have been generated by multiply attenuating the HIV virulence genes, for example, the genes Env, Vif, Vpr, Vpu and Nef are deleted, making the vector biologically safe.

Lentiviral vectors offer many advantages for gene therapy. Unless engineered to be non-integrating, lentiviral vectors integrate stably into chromosomes of target cells, permitting long-term expression of delivered transgenes. Further, they do not transfer viral genes thus avoiding the problem of generating transduced cells that can be destroyed by cytotoxic T cells. Further, they have a relatively large cloning capacity, sufficient for most envisioned clinical applications. Among retroviruses, lentiviruses have the unique ability to integrate their genome into the chromatin of nondividing cells. This is especially important in the context of gene-therapy for tissues, for example, in the hematopoietic system, the brain, liver, lungs, and muscle. For example, vectors derived from HIV-1 allow efficient in vivo and ex vivo delivery, integration and stable expression of transgenes into cells such as neurons, hepatocytes, and myocytes (Blomer et al., 1997; Kafri et al., 1997; Naldini et al., 1996; Naldini et al., 1998).

Lentiviral vectors are known in the art. For example, see Naldini et al. (1996) Science 272: 263-267; Zufferey et al. (1998) J Virol. 72: 9873-9880; Dull et al. (1998) J Virol. 72: 8463-8471; U.S. Pat. Nos. 6,013,516; 5,994,136. Generally, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems can include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences that facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. Additionally, the transfer plasmid can be designed with a deletion of the 3′ LTF, rendering the virus “self-inactivating” (SIN). See Dull et al. (1998) J. Virol. 72:8463-8471; Miyoshi et al. (1998) J. Virol. 72:8150-8157.

Third-generation systems also generally include two “packaging plasmids” and an “envelope plasmid.” The “envelope plasmid” generally encodes an Env gene operatively linked to a promoter. In at least one embodiment of a third-generation system, the Env gene is VSV-G and the promoter is the CMV promoter. The third-generation system uses two packaging plasmids, one encoding Gag and Pol and the other encoding Rev as a further safety feature—an improvement over the single packaging plasmid of so-called second-generation systems. Although safer, the third-generation system can be more cumbersome to use and result in lower viral titers due to the addition of an additional plasmid. Examplary packing plasmids include, without limitation, pMD2.G, pRSV-rev, pMDLG-pRRE, and pRRL-GOI.

In some instances, lentiviral vector systems rely on the use of a “packaging cell line.” In general, the packaging cell line is a cell line whose cells can produce infectious lentiviral particles when the transfer plasmid, packaging plasmid(s), and envelope plasmid are introduced into the cells. Various methods of introducing the plasmids into the cells can be used, including transfection or electroporation. In some cases, a packaging cell line is adapted for high-efficiency packaging of a lentiviral vector system into lentiviral particles.

As used herein, the term “lentiviral vector” means a nucleic acid that encodes a lentiviral cis nucleic acid sequence required for genome packaging. A lentiviral vector also can encode other cis nucleic acid sequences beneficial for gene delivery, including for example, cis sequences required for reverse transcription, proviral integration or genome transcription. A lentiviral vector performs transduction functions of a lentiviral vector. As such, the exact makeup of a vector genome will depend on the genetic material desired to be introduced into a target cell. Therefore, a vector genome can encode, for example, additional polypeptides or functions other than that required for packaging, reverse transcription, integration, or transcription. Such functions generally include coding for cis elements required for expression of a nucleic acid of interest. The lentiviral cis sequences or elements can be derived from a lentivirus genome or other virus or vector genome so long as the lentiviral vector genome can be packaged by a packaging cell line into a lentiviral particle and introduced into a target cell. In certain embodiments, the target cell for the lentiviral vector is an immune cell. In certain embodiments, the target immune cell is a T cell or NK cell. In certain embodiments, the target immune cell exists in a tumor microenvironment.

The lentiviral particles produced generally include an RNA genome (e.g., derived from a transfer plasmid), a lipid-bilayer envelope in which the Env protein is embedded, and other accessory proteins including integrase, protease, and matrix protein. As used herein, the term “lentiviral particle” means a viral particle that includes an envelope, has one or more characteristics of a lentivirus, and can invade a target host cell (e.g., a T cell or NK cell). Such characteristics can include, for example, infecting non-dividing host cells, transducing non-dividing host cells, infecting or transducing host immune cells, containing a lentiviral virion including one or more of the gag structural polypeptides p7, p24, and p17, containing a lentiviral envelope including one or more of the env encoded glycoproteins p41, p120, and p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev.

Lentiviral vectors can be used to encode T cell activation receptors. A “T cell activation receptor” means one or more transmembrane proteins that are configured to be expressed on the cell surface of transduced cells such that the T cell activation receptor provides a mitogenic signal to the transduced cell. A T cell activation receptor is used because the target cells, in certain cases, are T cells. The present methods can be adapted for use with other cell types by use of an activation receptor that retains activity in another cell type, for example, NK cells. T cell activation receptors useful here can include a signaling domain that is a cytokine receptor signaling domain, a co-stimulatory receptor signaling domain, a T cell receptor subunit signaling domain, a growth factor receptor signaling domain, or the like (e.g., as previously described in connection with the CAR compositions).

It can be advantageous, in some cases, to provide a means to target transduced cells to particular cells or tissues. Accordingly, the lentiviral vector can comprise (instead of or in addition to other genes) a polynucleotide encoding a CAR described herein.

As is known, lentiviral vectors can further comprise promoters and/or enhancers specific to T cells. In some cases, promoters can be used to control expression of the T cell activation receptor. Further, lentiviral vectors can include fusion glycoproteins (e.g., for pseudotyping purposes). See, e.g., Joglekar et al. (2017) Human Gene Therapy Methods 28:291-301. In certain embodiments, pseudo-typing a fusion glycoprotein or functional variant thereof facilitates targeting transduction of specific cell types including, without limitation, T cells.

In certain embodiments, vectors hereof can include the Woodchuck Hepatitis Virus Posttranscriptional Regulatory Element (wPRE) or a nucleic acid sequence substantially identical to wPRE. See U.S. Pat. No. 6,136,597; Lee et al. (2005) Exp Physiol. 90:33-37. Variants of the wPRE element with reduced size are known in the art. wPRE-O refers to a variant of wPRE with the intermediate size. In some embodiments, the wPRE sequence increases expression of genes delivered by such viral vectors.

In some cases, lentiviral vectors can comprise a polynucleotide sequence encoding the 2A peptide. The term “2A peptide” refers to a self-cleaving peptide configured to generate two or more proteins from a single open reading frame. 2A peptides are 18-22 residue-long viral oligopeptides that mediate “cleavage” of polypeptides during translation in eukaryotic cells. “2A peptide” can refer to peptides with various amino acid sequences. Detailed methodology for design and use of 2A peptides is provided by Szymczak-Workman et al. (2012) Cold Spring Harb. Protoc. 2012: 199-204.

In some embodiments, vector compositions are administered directly to the subject. In some embodiments, vector compositions are administered in conjunction with cytotoxic lymphocytes. In some embodiments, vector compositions and cytotoxic lymphocytes are separately administered. In some embodiments, cytotoxic lymphocytes are activated and transduced in vivo by administered vector compositions.

### Combinations, Compositions, and Methods for Treating Cancer

Combinations and compositions for treating a cancer (e.g., a solid tumor) are also provided. As used herein, the term “combination” generally refers to any product comprising more than one ingredient, including one or more of the compounds described herein (e.g., a SMDC or a pharmaceutically acceptable salt of the foregoing). It is to be understood that the compositions described herein can be prepared from isolated compounds or from salts, solutions, hydrates, solvates, and other forms of the compounds. It is appreciated that certain functional groups, such as the hydroxy, amino, and like groups, can form complexes with water and/or various solvents, in the various physical forms of the compounds. It will also be understood that, in certain circumstances, the compounds (and compositions comprising the compounds) can be prepared from various amorphous, non-amorphous, partially crystalline, crystalline, and/or other morphological forms of the compounds, and the compositions can be prepared from various hydrates and/or solvates of the compounds. Accordingly, pharmaceutical compositions that recite the compounds described herein include each of, or any combination of, or individual forms of, the various morphological forms and/or solvate or hydrate forms of the compounds.

A combination for treating cancer comprises one or more compounds comprising the SMDCs hereof (or pharmaceutically acceptable salts thereof) and/or one or more compositions hereof, and a composition comprising cytotoxic lymphocytes expressing a CAR or a vector comprising a promoter operatively linked to a nucleic acid sequence encoding the CAR.

Compounds and compositions can be administered in unit dosage forms and/or compositions containing one or more pharmaceutically acceptable carriers, adjuvants, diluents, excipients, and/or vehicles, and combinations thereof. The term “administering,” and its formatives, generally refer to any and all means of introducing the compounds and compositions described herein (e.g., the CAR-expressly cytotoxic lymphocyte compositions and/or SMDC compounds or compositions) to a cell, tissue, organ, or biological fluid of a subject.

Administration of the compounds and compositions as salts may be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable.

As used herein, a “subject” is a mammal, preferably a human, but it can also be a non-human animal (including, without limitation, a laboratory, an agricultural, a domestic, or a wild animal). Thus, the methods, compounds, and compositions described herein are applicable to both human and veterinary disease and applications. In various aspects, the subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, or a goat, or a wild animal in captivity such as a bear, a panda, a lion, a tiger, a leopard, an elephant, a zebra, a giraffe, a gorilla, a dolphin, or a whale. In certain embodiments, subjects are “patients,” i.e., living humans or animals that are receiving medical care for a disease or condition, which includes persons or animals with no defined illness who are being evaluated for signs of pathology. In certain embodiments, subjects that can be addressed using the methods hereof include subjects identified or selected as having or being at risk for having cancer. Such identification and/or selection can be made by clinical or diagnostic evaluation.

The compounds and compositions can be formulated as pharmaceutical compositions and/or administered to a subject, such as a human patient, in a variety of forms adapted to the chosen route of administration. Indeed, the adaptor compound, or the pharmaceutically acceptable salt thereof, or the activity modifying compound, or the pharmaceutically acceptable salt thereof, or the CAR-expressing cytotoxic lymphocyte composition, or the vector composition (including, for example, the lentiviral particles hereof) can be administered to a subject using any suitable method known in the art. In one aspect, the SMDC compound, or the pharmaceutically acceptable salt thereof can be administered in unit dosage forms and/or formulations containing conventional nontoxic pharmaceutically acceptable carriers, adjuvants, and vehicles.

Further, the SMDC compound(s), or the pharmaceutically acceptable salt(s) thereof, or the CAR-expressing cytotoxic lymphocyte composition, or the vector composition as described herein can be administered directly into the blood stream, into muscle, or into an internal organ. In various embodiments, suitable routes for such parenteral administration include intravenous, intraarterial, intraperitoneal, intrathecal, epidural, intracerebroventricular, intraurethral, intrastemal, intracranial, intratumoral, intramuscular and subcutaneous delivery. In one embodiment, means for parenteral administration include needle (including microneedle) injectors, needle-free injectors and infusion techniques. It will be appreciated that the compounds and compositions hereof can be formulated for the desired administration modality as well.

For example, parenteral formulations are typically aqueous solutions and can contain carriers or excipients such as salts, carbohydrates and buffering agents (preferably at a pH of from 3 to 9), but can also be formulated, where suitable, as a sterile non-aqueous solution or as a dried form to be used in conjunction with a suitable vehicle such as sterile, pyrogen-free water or sterile saline. In other embodiments, any of the liquid formulations described herein can be adapted for parenteral administration. The preparation under sterile conditions, by lyophilization to produce a sterile lyophilized powder for a parenteral formulation, can readily be accomplished using standard pharmaceutical techniques well-known to those skilled in the art.

The pharmaceutical dosage forms of the SMDC compound(s) that are suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active component in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

Useful dosages of the compounds can be determined by comparing their in vitro activity and the in vivo activity in animal models. Methods of the extrapolation of effective dosages in mice and other animals to human subjects are known in the art. Indeed, the dosage of the SMDC compound can vary significantly depending on the condition of the subject, the cancer type being treated, how advanced the pathology is, the route of administration of the compound and tissue distribution, and the possibility of co-usage of other therapeutic treatments (such as radiation therapy or additional drugs in combination therapies). The amount of the compositions and/or compound(s) required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. “Therapeutically effective amount,” “therapeutically effective dose,” “therapeutically effective,” or “prophylactically effective amount” is defined as (unless specifically stated otherwise) an amount of a conjugate (e.g., the SMDC) or pharmaceutical composition that, when administered either one time or over the course of a treatment cycle affects the health, well-being or mortality of a subject (e.g., and without limitation, delays the onset of and/or reduces the severity of one or more of the symptoms associated with the cancer).

In various embodiments, the transduced CAR-expressing cytotoxic lymphocytes administered to the subject can comprise from about 1×10⁵ to about 1×10¹⁵ or 1×10⁶ to about 1×10¹⁵ transduced CAR-T cells. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷ about 1×10⁶ to about 3×10⁷, about 1×10⁶ to about 1.5×10⁷, about 1×10⁶ to about 1×10⁷ about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ CAR-T cells can be administered to the subject. In one aspect, in any embodiment described herein, a single dose or multiple doses of the CAR-expressing cytotoxic lymphocytes can be administered to the subject. In any of the embodiments described in this paragraph, the CAR-expressing cytotoxic lymphocyte dose can be in numbers of CAR-expressing cytotoxic lymphocytes per kg of subject body weight. In any embodiment described herein, the CAR-expressing cytotoxic lymphocytes can be administered before or after the SMDC compound(s), or the pharmaceutically acceptable salt thereof.

In other embodiments, the dose of the CAR-expressing cytotoxic lymphocytes administered to the subject in the CAR-expressing cytotoxic lymphocyte composition is selected from the group consisting of about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, and about 15 million of the CAR-expressing cytotoxic lymphocytes. In these embodiments, the CAR-expressing cytotoxic lymphocyte dose can be in numbers of CAR-expressing cytotoxic lymphocytes per kg of subject body weight.

Therapeutically effective or prophylactically effective amounts or doses of the SMDC compound (or pharmaceutically acceptable salts thereof) can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. One of skill in the art will readily appreciate that the dose may vary within the various ranges provided above based on the factors noted above and may be at the physician's discretion.

In another embodiment, the conjugate can be administered in a therapeutically or prophylactically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m2, from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m2 to about 2.0 mg/m².

For example, in other embodiments, the therapeutically effective or prophylactically effective amount or dose of the SMDC, or the pharmaceutically acceptable salt thereof, can range, for example, from about 50 nmoles/kg to about 3,000 nmoles/kg of subject body weight, about 50 nmoles/kg to about 2,800 nmoles/kg about 50 nmoles/kg to about 2,600 nmoles/kg about 50 nmoles/kg to about 2,400 nmoles/kg about 50 nmoles/kg to about 2,200 nmoles/kg about 50 nmoles/kg to about 2,100 nmoles/kg about 50 nmoles/kg to about 2,000 nmoles/kg, about 50 nmoles/kg to about 1,000 nmoles/kg, about 50 nmoles/kg to about 900 nmoles/kg, about 50 nmoles/kg to about 800 nmoles/kg, about 50 nmoles/kg to about 700 nmoles/kg, about 50 nmoles/kg to about 600 nmoles/kg, about 50 nmoles/kg to about 500 nmoles/kg, about 50 nmoles/kg to about 400 nmoles/kg, about 50 nmoles/kg to about 300 nmoles/kg, about 50 nmoles/kg to about 200 nmoles/kg, about 50 nmoles/kg to about 100 nmoles/kg, about 100 nmoles/kg to about 300 nmoles/kg, about 100 nmoles/kg to about 500 nmoles/kg, about 100 nmoles/kg to about 1,000 nmoles/kg, about 100 nmoles/kg to about 2,000 nmoles/kg of subject body weight. In other embodiments, the dose may be about 1 nmoles/kg, about 5 nmoles/kg, about 10 nmoles/kg, about 20 nmoles kg, about 25 nmoles/kg, about 30 nmoles/kg, about 40 nmoles/kg, about 50 nmoles/kg, about 60 nmoles/kg, about 70 nmoles/kg, about 80 nmoles/kg, about 90 nmoles/kg, about 100 nmoles/kg, about 150 nmoles/kg, about 200 nmoles/kg, about 250 nmoles/kg, about 300 nmoles/kg, about 350 nmoles/kg, about 400 nmoles/kg, about 450 nmoles/kg, about 500 nmoles/kg, about 600 nmoles/kg, about 700 nmoles/kg, about 800 nmoles/kg, about 900 nmoles/kg, about 1000 nmoles/kg, about 2,000 nmoles/kg, about 2,500 nmoles/kg or about 3,000 nmoles/kg of body weight of the subject. In yet other embodiments, the dose may be about 0.1 nmoles/kg, about 0.2 nmoles/kg, about 0.3 nmoles/kg, about 0.4 nmoles kg, or about 0.5 nmoles/kg, about 0.1 nmoles/kg to about 1000 nmoles/kg, about 0.1 nmoles/kg to about 900 nmoles/kg, about 0.1 nmoles/kg to about 850 nmoles/kg, about 0.1 nmoles/kg to about 800 nmoles/kg, about 0.1 nmoles/kg to about 700 nmoles/kg, about 0.1 nmoles/kg to about 600 nmoles/kg, about 0.1 nmoles/kg to about 500 nmoles/kg, about 0.1 nmoles/kg to about 400 nmoles/kg, about 0.1 nmoles/kg to about 300 nmoles/kg, about 0.1 nmoles/kg to about 200 nmoles/kg, about 0.1 nmoles/kg to about 100 nmoles/kg, about 0.1 nmoles/kg to about 50 nmoles/kg, about 0.1 nmoles/kg to about 10 nmoles/kg, or about 0.1 nmoles/kg to about 1 nmoles/kg of body weight of the subject. In other embodiments, the dose may be about 0.3 nmoles/kg to about 1000 nmoles/kg, about 0.3 nmoles/kg to about 900 nmoles/kg, about 0.3 nmoles/kg to about 850 nmoles/kg, about 0.3 nmoles/kg to about 800 nmoles/kg, about 0.3 nmoles/kg to about 700 nmoles/kg, about 0.3 nmoles/kg to about 600 nmoles/kg, about 0.3 nmoles/kg to about 500 nmoles/kg, about 0.3 nmoles/kg to about 400 nmoles/kg, about 0.3 nmoles/kg to about 300 nmoles/kg, about 0.3 nmoles/kg to about 200 nmoles/kg, about 0.3 nmoles/kg to about 100 nmoles/kg, about 0.3 nmoles/kg to about 50 nmoles/kg, about 0.3 nmoles/kg to about 10 nmoles/kg, or about 0.3 nmoles/kg to about 1 nmoles/kg of body weight of the subject.

In various other embodiments, the therapeutically effective or prophylactically effective dose of the SMDC compound, or the pharmaceutically acceptable salt thereof, can range from, for example, about 10 nmoles/kg to about 10,000 nmoles/kg, from about 10 nmoles/kg to about 5,000 nmoles/kg, from about 10 nmoles/kg to about 3,000 nmoles/kg, about 10 nmoles/kg to about 2,500 nmoles/kg, about 10 nmoles/kg to about 2,000 nmoles/kg, about 10 nmoles/kg to about 1,000 nmoles/kg, about 10 nmoles/kg to about 900 nmoles/kg, about 10 nmoles/kg to about 800 nmoles/kg, about 10 nmoles/kg to about 700 nmoles/kg, about 10 nmoles/kg to about 600 nmoles/kg, about 10 nmoles/kg to about 500 nmoles/kg, about 10 nmoles/kg to about 400 nmoles/kg, about 10 nmoles/kg to about 300 nmoles/kg, about 10 nmoles/kg to about 200 nmoles/kg, about 10 nmoles/kg to about 150 nmoles/kg, about 10 nmoles/kg to about 100 nmoles/kg, about 10 nmoles/kg to about 90 nmoles/kg, about 10 nmoles/kg to about 80 nmoles/kg, about 10 nmoles/kg to about 70 nmoles/kg, about 10 nmoles/kg to about 60 nmoles/kg, about 10 nmoles/kg to about 50 nmoles/kg, about 10 nmoles/kg to about 40 nmoles/kg, about 10 nmoles/kg to about 30 nmoles/kg, about 10 nmoles/kg to about 20 nmoles/kg, about 200 nmoles/kg to about 900 nmoles/kg, about 200 nmoles/kg to about 800 nmoles/kg, about 200 nmoles/kg to about 700 nmoles/kg, about 200 nmoles/kg to about 600 nmoles/kg, about 200 nmoles/kg to about 500 nmoles/kg, about 250 nmoles/kg to about 600 nmoles/kg, about 300 nmoles/kg to about 600 nmoles/kg, about 300 nmoles/kg to about 500 nmoles/kg, or about 400 nmoles/kg to about 600 nmoles/kg.

In various other embodiments, the therapeutically effective or prophylactically effective dose of the SMDC compound, or the pharmaceutically acceptable salt thereof, can range from, for example, about 1 nmoles/kg to about 10,000 nmoles/kg, from about 1 nmoles/kg to about 5000 nmoles/kg, from about 1 nmoles/kg to about 3000 nmoles/kg, about 1 nmoles/kg to about 2500 nmoles/kg, about 1 nmoles/kg to about 2000 nmoles/kg, about 1 nmoles/kg to about 1000 nmoles/kg, about 1 nmoles/kg to about 900 nmoles/kg, about 1 nmoles/kg to about 800 nmoles/kg, about 1 nmoles/kg to about 700 nmoles/kg, about 1 nmoles/kg to about 600 nmoles/kg, about 1 nmoles/kg to about 500 nmoles/kg, about 1 nmoles/kg to about 400 nmoles/kg, about 1 nmoles/kg to about 300 nmoles/kg, about 1 nmoles/kg to about 200 nmoles/kg, about 1 nmoles/kg to about 150 nmoles/kg, about 1 nmoles/kg to about 100 nmoles/kg, about 1 nmoles/kg to about 90 nmoles/kg, about 1 nmoles/kg to about 80 nmoles/kg, about 1 nmoles/kg to about 70 nmoles/kg, about 1 nmoles/kg to about 60 nmoles/kg, about 1 nmoles/kg to about 50 nmoles/kg, about 1 nmoles/kg to about 40 nmoles/kg, about 1 nmoles/kg to about 30 nmoles/kg, or about 1 nmoles/kg to about 20 nmoles/kg.

In another embodiment, from about 20 μg/kg body weight to about 3 mg/kg body weight of the SMDC compound, or the pharmaceutically acceptable salt thereof, can be administered to the subject. In another aspect, amounts can be from about 0.2 mg/kg body weight to about 0.4 mg/kg body weight or can be about 50 μg/kg body weight.

Unless otherwise specified, in all the dosage embodiments set forth herein, “kg” is kilograms of body weight of the subject.

The total therapeutically effective or prophylactically effective amount of the conjugate (e.g., SMDC) can be administered in single or divided doses and can, at the practitioner's discretion, fall outside of the typical ranges given herein.

The timing between the administration of CAR-expressing cytotoxic lymphocytes and the SMDC compound and/or composition can vary widely depending on factors that include the type of CAR-expressing cytotoxic lymphocytes being used, the binding specificity of the CAR, the identity of the targeting moiety/ligand of the SMDC, the identity of the cancer, the location in the subject of the cancer, the means used to administer to the subject the CAR-expressing cytotoxic lymphocytes and the SMDC compound, or the pharmaceutically acceptable salt thereof, and the health, age, and weight of the subject.

In at least one embodiment, the SMDC compound(s) or the pharmaceutically acceptable salts thereof can be administered before or after the CAR-expressing cytotoxic lymphocytes (or composition thereof), such as within about 3, 6, 9, 12, 15, 18, 21, 24, 27, 30, 33, 36, 39, 42, 45, 48, or 51 hours, or within about 0.5, 1, 1.5, 2, 2.5, 3, 4 5, 6, 7, 8, 9, 10 or more days. In another embodiment, the SMDC compound(s) or pharmaceutically acceptable salts thereof can be administered to the subject at the same time as the CAR-expressing cytotoxic lymphocytes composition, but in different formulations, or in the same formulation.

Any applicable dosing schedule known in the art can be used for administration of the SMDC compound(s), or the pharmaceutically acceptable salt(s) thereof, or for the CAR-expressing cytotoxic lymphocyte composition. For example, once per day dosing (a.k.a. qd), twice per day dosing (a.k.a. bid), three times per day dosing (a.k.a. tid), twice per week dosing (a.k.a. BIW), three times per week dosing (a.k.a. TIW), once weekly dosing, and the like, can be used. In one aspect, the dosing schedule selected can take into consideration the concentration of the compounds/compositions being administered (including, for example, the number of CAR-expressing cytotoxic lymphocytes administered) to regulate the cytotoxicity of the CAR-expressing cytotoxic lymphocyte composition and to control any potential adverse effects.

Also provided is a method of treating a patient for cancer. The method comprises administering any of the above-described combination cancer therapy (including, without limitation, administration of any of the combinations, compounds and compositions hereof) to the patient, whereupon the patient is treated for cancer.

In the methods described herein, the cancer can additionally be imaged prior to administration to the subject of the SMDC compound(s), or the pharmaceutically acceptable salts thereof, or the CAR-expressing cytotoxic lymphocyte composition. The cancer additionally, or alternatively, can be imaged during or after administration to assess metastasis, for example, and the efficacy of treatment. For example, imaging can occur by positron emission tomography (PET) imaging, magnetic resonance imaging (MRI), or single-photon-emission computed tomography (SPECT)/computed tomography (CT) imaging. The imaging method can be any suitable imaging method known in the art.

The cancer can be any cancer. “Cancer” has its plain and ordinary meaning when read in light of the specification and can include, but is not limited to, a group of diseases involving abnormal cell growth with the potential to invade or spread (i.e., metastasize) to other parts of the body. Examples include, but are not limited to, a cancer of the brain, thyroid, lung, pancreas, kidney, stomach, gastrointestinal stroma, endometrium, breast, cervix, ovary, colon, prostate, leukemias, lymphomas, other blood-related cancers, or head and neck cancer. In certain embodiments, the cancer being treated is a tumor. In certain embodiments, the cancer is malignant.

In some aspects of these embodiments, the cancer is a folate receptor-expressing cancer, for example and without limitation, a folate receptor α-expressing cancer. In other embodiments, the cancer is a folate receptor β-expressing cancer.

In the compounds, compositions, combinations, and methods, all embodiments of the at least one SMDC (including, without limitation, the drug moiety or pharmaceutically acceptable salt thereof, and/or the ligand/targeting moiety thereof), the CAR-expressly cytotoxic lymphocyte compositions, and the vector compositions are applicable, including, but not limited to, the linker embodiments.

### EXAMPLES

The following examples serve to illustrate the present disclosure. The examples are not intended to limit the scope of the claimed invention in any way.

### Example 1: Confirmation of Folate Receptor Level of 4T1 CT26, and EMT6 Cell Lines in the Folate-Deficient Environment

To establish that the selective repolarization of TAMs and MDSCs in a TME by a folate receptor-targeted TLR7 agonist (FA-TLR7-1A; Compound 2) enhanced the anti-solid tumor efficacy of CAR-T cells, cancer cell lines devoid of both folate receptors and TLR7s were identified (so that Compound 2 would not directly alter cancer cell behavior). To identify such cancer cell lines, several commonly employed syngeneic tumor cell lines were screened for folate receptor expression (FR) by evaluating their ability to bind a folate-fluorescein conjugate. CT26, 4T1, and EMT6 were all found to lack detectable FR (as compared with positive control in L1210A cells).

To confirm that the selected cancer cells were indeed FR⁻ in the folate-deficient environment, the identified mouse cancer cell lines 4T1, CT26, and EMT6 cells, and FR⁺ mouse cancer cell line L1210A, were cultured in folic acid-free Roswell Park Memorial Institute (RPMI) 1640 (Gibco, Ireland) containing 10% heat-inactivated fetal calf serum and 1% penicillin-streptomycin in 5% CO₂ at 37° C. for a week. The cells were then detached by 1 μM ethylenediaminetetraacetic acid (EDTA)+1 μM egtazic acid (EGTA) and stained with folate-fluorescein isothiocyanate (FITC) (10 nM) for 1 hour at room temperature. All the samples were then washed three times with PBS and analyzed by flow-cytometry for folate-FITC binding. The stained samples were protected from light during the entire process. This inability to bind folate-FITC is commonly used to identify cell lines that do not express a functional folate receptor.

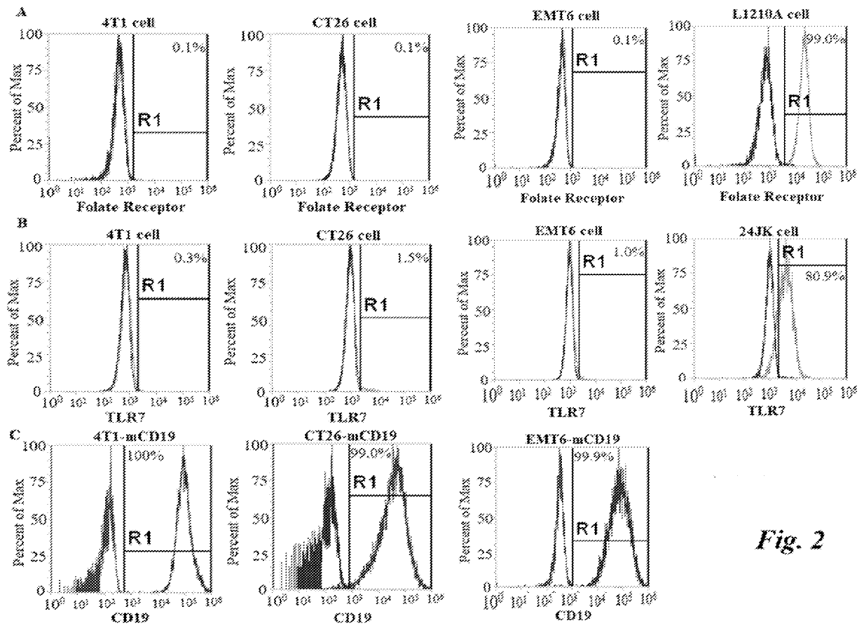

As seen in the flow cytometry histograms of FIG. 2A, mouse cancer cell lines CT26, 4TI, and EMT6 were all identified as lacking detectable FR (as compared with positive control in L1210A cells), even after culturing in folate-deficient medium to try to induce FR upregulation.

### Example 2: TLR7 Expression Levels in 4T1 CT26, and EMT6 Cell Lines

The cell lines were examined by flow cytometry using an antibody to murine TLR7 to determine if the identified cell lines express TLR7. Each of the 4T1, CT26, and EMT6 cells were detached using 0.25% trypsin and washed once with PBS. The cell lines were fixed and permeabilized with Cyto-Fast™ Fix/Perm Buffer Set (BioLegend, San Diego, CA). TLR7⁺ mouse cancer cell line 24JK was treated with the same steps as a positive control.

All four cell lines were incubated with anti-mouse CD16/CD32 (BioLegend, San Diego, CA) in 100 μl volume for 15 minutes at room temperature. The cell lines were then incubated with anti-mouse TLR7-PE antibody (BD Biosciences, Franklin Lakes, NJ) for 20 minutes at room temperature in the dark. All samples were then washed two times with 1 ml PBS and analyzed by flow cytometry. The stained samples were protected from light during the whole process. As seen in FIG. 2B, none of the cell lines expressed TLR7 (as compared with positive control 24JK cells), indicating that neither free TLR7-la nor a FA-TLR7-1A conjugate (e.g., Compound 2) directly activated the above cancer cell lines.

### Example 3: Transduction of Syngeneic Cancer Cell Lines

A cell line was created that would normally be treatable with a classical anti-CD19 CAR-T cell therapy. CT26, 4T1, and EMT6 cells were first transduced with mouse CD19 linked with green fluorescent protein (GFP) using a lentiviral vector. HEK-293T cells and Lipofectamine 3000 were used to produce the lentivirus, and polybrene (8 μg/mL) was used during transduction. The transduced cells were then sorted by flow cytometry to select clones that were found to express high levels of GFP and mCD19 (see FIG. 2C). The growth of each transduced cell line was then examined in immune competent syngeneic mice to identify cell lines with strong tumor-forming potential in vivo.

Flow cytometry sorted CD19 expressing 4T1 cells were diluted to 1 cell/200p1 in RPMI 1640 media and seeded into 96 well plants with 100 μl/well. Thereafter, the wells with only one colony were detached with 0.25% trypsin and transferred from one well to one well into 96 Well Black/Clear Bottom Plates. Each well in the plates was then screened using Opera Phenix™ High Content Screening System (PerkinElmer, Waltham, MA) to select the clones with high GFP expression. The selected single cell clones were then expanded to around 1×10⁷ cells and stained with anti-mouse CD19-PE antibody to compare the mCD19 expression level. The cells from the single cell clone with the highest mean fluorescence intensity (MFI) were aliquoted and stored in liquid nitrogen for later studies. Single cell clones of CD19-expressing CT26 and EMT6 cells were produced using the same procedure. The expression levels of CD19 on all three selected CD19-expressing murine cancer cell lines were checked by flow cytometry after one month of in vitro culturing.

While all cell lines stably expressed mCD19, the mCD19-expressing 4T1 cell clone (4T1-mCD19) was elected for all further studies as, out of the three cell lines studied, 4T1 formed the most aggressive tumors in mice.

### Example 4: In Vivo Cytotoxicity of Anti-mCD19 CAR-T Cells Against CD19⁺ Cancer Cells

4T1-mCD19 was co-cultured overnight in 96-well plates with either a first set of anti-CD19 CAR-T cells or a second set of non-transduced T cells to assess the in vitro cytotoxicity of anti-mCD19 CAR-T cells against 4T1-mCD19⁺ cancer cells.

The first set of T cells was transduced with a retroviral vector to express anti-mCD19 CAR (MSGV1-1D3-28Z.1-3 mut). Mice T cells were isolated from the spleens of Balb/c mice (i.e., the mouse strain from which 4T1 cells are derived) through negative selection (EasySep™ Mouse T Cell Isolation Kit, STEMCELL Technologies, Vancouver, CA). After isolation, mouse T cells were activated with anti-CD3/CD28-conjugated Dynabeads for 24 hours. The activated T cells were then transferred into RetroNectin-coated plates for transduction. Second transduction was performed one day after the first transduction to improve the expression of anti-mouse CD19 CAR on mouse T cells. Since the anti-mouse CD19 scFv was derived from an antibody produced by rats, an anti-rat IgG F(ab′)₂ fragment conjugated with Alexa Fluor 594 was used to stain the transduced and non-transduced mice T cells.

The effector cells to target cells ratios included were 1:1, 2:1, and 5:1. The next day after co-culturing, all wells were gently washed once with PBS to remove most of the suspended cells, 50 μl of 0.25% trypsin were added to each well, and the μlate was incubated at 37° C. for 20 minutes. All cells in each well were then pipetted and collected. Each well was washed twice with RPMI 1640 media containing 10% FBS to collect all the cells and neutralize the trypsin. From co-culturing to washing, all the wells had the same total volume.

Fifteen minutes before running flow cytometry for cell counting, 1 μl 7-Aminoactinomycin D (7-AAD) was added to each well to distinguish living and dead cells. The T cells and the 4T1-mCD19 cells were distinguished by size and GFP level. The speed and volume of each sample taken by flow cytometer were set to be the same to compare the remaining living 4T1-mCD19 cells in each well.

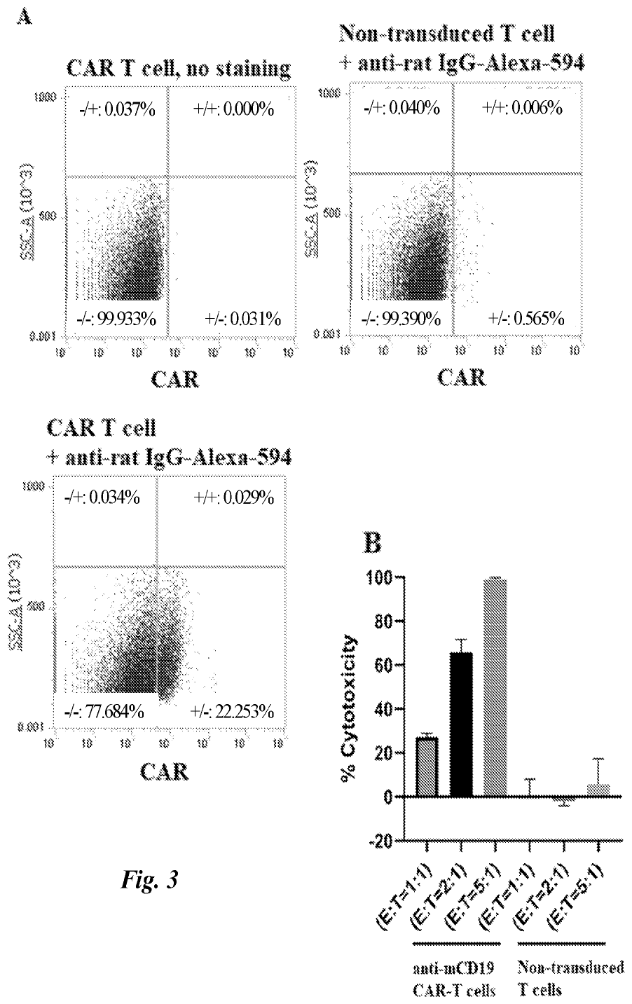

As shown in FIG. 3A, murine anti-CD19 CAR-T cells were routinely generated with about 20% efficiency, and their ability to kill 4T1-mCD19 cells reached 100% efficacy at an effector to tumor cell ratio of 5:1 (see FIG. 3B). In contrast, non-transduced T cells of the second set lacking an anti-CD19 CAR displayed insignificant cytotoxicity against the same tumor cell clones.

### Example 5: In Vivo Cytotoxicity of Anti-CD19 CAR-T Cells

To evaluate the ability of anti-CD19 CAR-T cells to kill CD19⁺ tumor cells in vivo, 5×10⁴ of syngeneic 4T1-mCD19 breast cancer cells (single cell clone) were injected subcutaneously into Balb/c mice, and the mice were allowed to form solid tumors. About 1 million CAR-T cells in a mixture also containing approximately 4 million non-transduced murine T cells were then infused into two parallel cohorts, with one such cohort also treated five days per week with a folate receptor-targeted TLR7 agonist (FA-TLR7-1A; Compound 2) and the other such cohort treated with saline. Each 4T1 tumor's growth was assessed during the treatment period for each cohort (i.e., in the presence and absence of the infused CAR-T cell mixture and/or the FA-TLR7-1A/Compound 2).

One day before CAR-T infusion, when the average tumor size reached 50 mm³, 4 Gy total body irradiation (TBI) was performed on each mouse for lymphodepletion. The treatment with Compound 2 or PBS (saline) was started when the average tumor size reached 50 mm³ and continued five times per week until the end of the study as noted above. The mice were arranged into the following cohorts for comparison: 1) treated with both CAR-T cells and Compound 2; 2) treated with anti-CD19 CAR-T cells only to quantitate the effect of anti-CD19 CAR-T cells alone on 4T1-mCD19 tumor growth; 3) treated with Compound 2 only; or 4) treated with PBS.

To check the expression of mouse CD19 on the 4T1 cells at the end of the study, the digested cells were stained with Zombie Violet (BioLegend, San Diego, CA) and antibodies for flow cytometry analysis. After Zombie Violet staining, the digested cells were stained with anti-mouse EpCAM-APC (BioLegend, San Diego, CA) and anti-mouse CD19-PE (BioLegend, San Diego, CA), incubated on ice for 20 minutes, and washed with PBS two times. All samples were incubated with anti-mouse CD16/CD32 for 20 minutes on ice before being stained with antibodies. Further, the cells isolated from homogenous 4T1-mCD19 tumors treated with or without CAR-T cells were cultured in vitro to check the potential change of the CD19 expression level on isolated 4T1-mCD19 cells.

To determine whether either the CAR-T cell therapy or the addition of Compound 2 might have caused significant systemic toxicity, the weights of all mice were recorded over the entire course of the treatment.

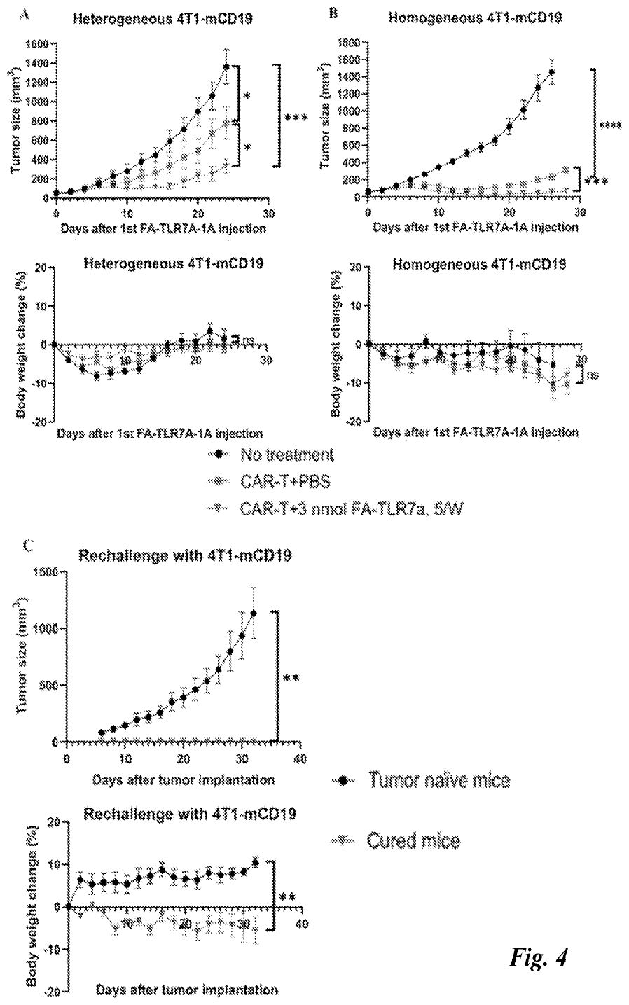

As shown in FIG. 4A, treatment of the tumor-bearing mice with murine CAR-T cells significantly suppressed tumor proliferation with little or no accompanying animal weight loss. Additionally, as shown in the lowest curve of FIG. 4B, concurrent administration of the TAM/MDSC-targeted immune stimulant (Compound 2) significantly improved the CAR-T cell potency, with 4 of the 9 treated mice exhibiting a complete response that was maintained for 30 additional days without further therapy. The mice treated with both CAR-T cell immunotherapy and Compound 2 experienced complete tumor cell eradication.

Rechallenge of these cured mice after the additional 30 days with an identical inoculum of 4T1-mCD19 cells again yielded mice with no detectable tumors (see FIG. 4C).

The cured mice were rechallenged by injecting 5×10⁴ 4T1-mCD19 cells into the left (contralateral) flank of each cured mice. A cohort of tumor naïve Balb/c mice treated with the same number of 4T1-mCD19 cells was used as a control. Again, after an additional 30 days, the rechallenged mice did not have any detectable tumors (FIG. 4C), and systemic toxicity (or off-target toxicity) was not identified. This supports that the complete responders were cured of their cancers and that during these treatments the mice developed a sustained immunity to the 4T1-mCD19 cancer cells.

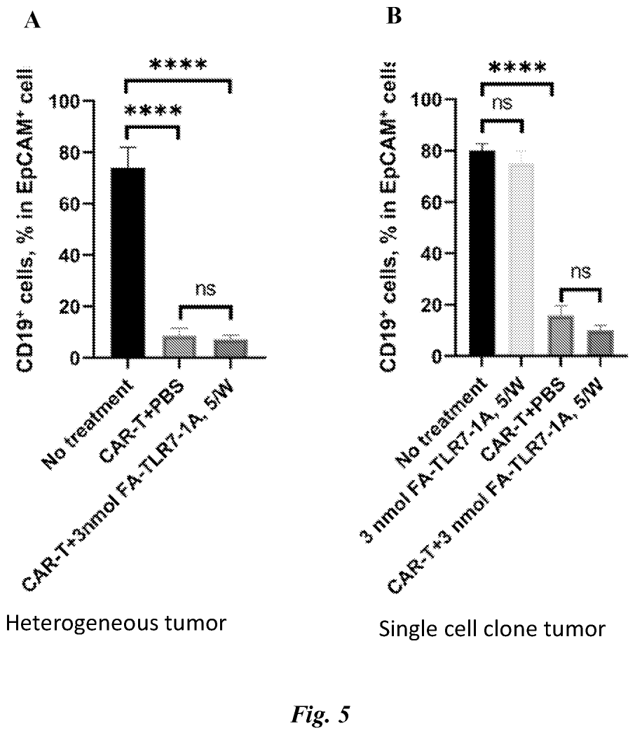

Examination of the residual tumor cells in the 5 mice that did not experience complete cures revealed that the resistant cancer cells all lacked the CD19 antigen (see FIG. 5A), suggesting their failure to respond completely to the therapy derived from their ability to suppress expression of CD19. To prevent the unwanted selection for mCD19⁻ cancer cells, single 4T1-mCD19 cells were cloned out and implanted into Balb/c mice to enable tumor growth. As shown in FIG. 5B, infusion of these mice solely with the same anti-CD19 CAR-T cells promoted an improved suppression of tumor growth, but not complete eradication of the tumor.

To further characterize the impact of co-administration of Compound 2 and CAR therapy, the weights of all animals treated with CAR therapy (both with and without concurrent treatment with Compound 2) were monitored. As seen in the lower panels of both FIGS. 4A and 4B, there were no significant differences in weight loss among the treated and untreated cohorts suggesting: (1) little (if any) free TLR7-la was released and available for systemic activation of immune cells; and (2) co-administration of Compound 2 can improve CART-T cell therapy without imposing significant toxicity (e.g., off-target toxicity).

### Example 6: Effect of Compound 2 Dosing Frequency

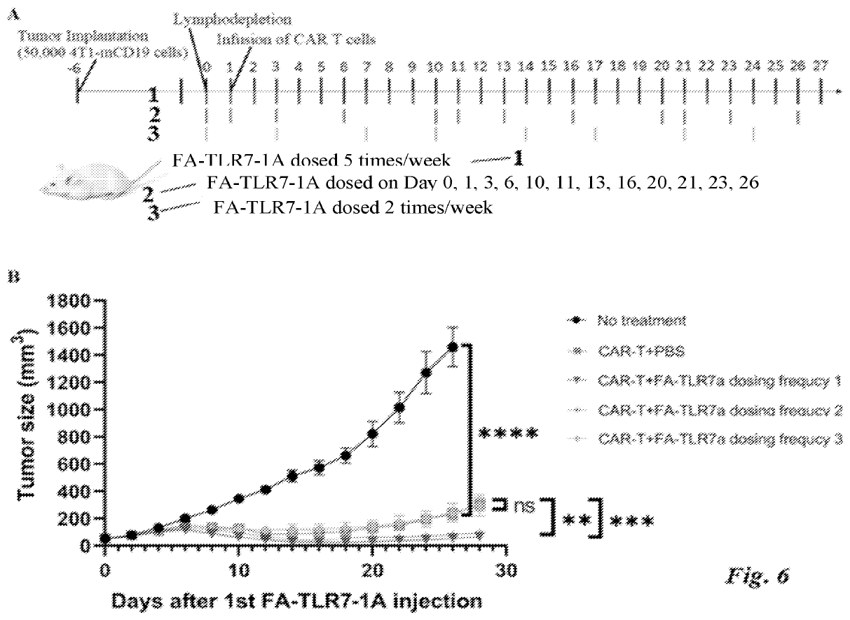

To optimize dosing frequency of Compound 2 to augment CAR therapy in solid tumors, different dosing frequencies of Compound 2 were applied to CAR-T treated Balb/c mice with 4T1-mCD19 (single cell clone) tumors. The treatment started when the average tumor size reached 50 mm³, and the dosing frequencies ranged from 2 times per week to 5 times per week (see FIG. 6A). The dosing frequency was also optimized in an antigenically heterogeneous tumor model by using flow cytometry sorted different clones of CD19-expressing 4T1 cells. Thereafter, the dosing regimen was studied to determine whether the 5 times per week Compound 2 dosing schedule was required for maximal enhancement of the CAR-T cell activity.

The efficacies of several related FA-TLR7 dosing frequencies on CAR-T cell efficacy were compared. As shown in FIG. 6B, no significant difference was observed between mice treated either 3 or 5 times per week with 3 nmol Compound 2; however, reducing the dosing frequency to only 2 times per week led to a reduction in potency. Based on this data, further studies were performed on mice treated 5 times per week with 3 nmoles FA-Compound 2 per dose and the data supports that a 5 times per week Compound 2 dosing schedule can be optimal for maximum enhancement of the CAR-T cell activity when co-administered.

### Example 7: Evaluation of Combination Therapy with Different Tumor Sizes

Other studies have indicated that the immunosuppressive properties of solid tumors can change with tumor size, where the properties of infiltrating TAMs and MDSCs in late-stage cancers are different from the ones in early-stage cancers. It has been reported that the TAMs in late-stage breast cancers are more protumoral, while the TAMs in early-stage breast cancers have anti-tumor effects. Moreover, the percentage of infiltrated MDSCs can be elevated in late-stage tumors as compared to early-stage tumors. Accordingly, the efficacy of combination therapy comprising Compound 2 and CAR-therapy on different sized 4T1 tumors was evaluated.

5×10⁴ 4T1-mCD19 cells (single cell clone) were injected subcutaneously into each Balb/c mouse. One day before CAR-T cell infusion, 4 Gy TBI was performed on each mouse for lymphodepletion.

To analyze the suppression effects of anti-CD19 CAR-T cells for different tumor sizes, an initial dose of Compound 2 or PBS was started when the average tumor size reached 50, 90, or 130 mm³ and continued 5 times per week until the end of the study. Each cohort was treated with 1) a combination therapy comprising both CAR-T cells and Compound 2; 2) CAR-T cells only; 3) Compound 2 only; or 4) PBS.

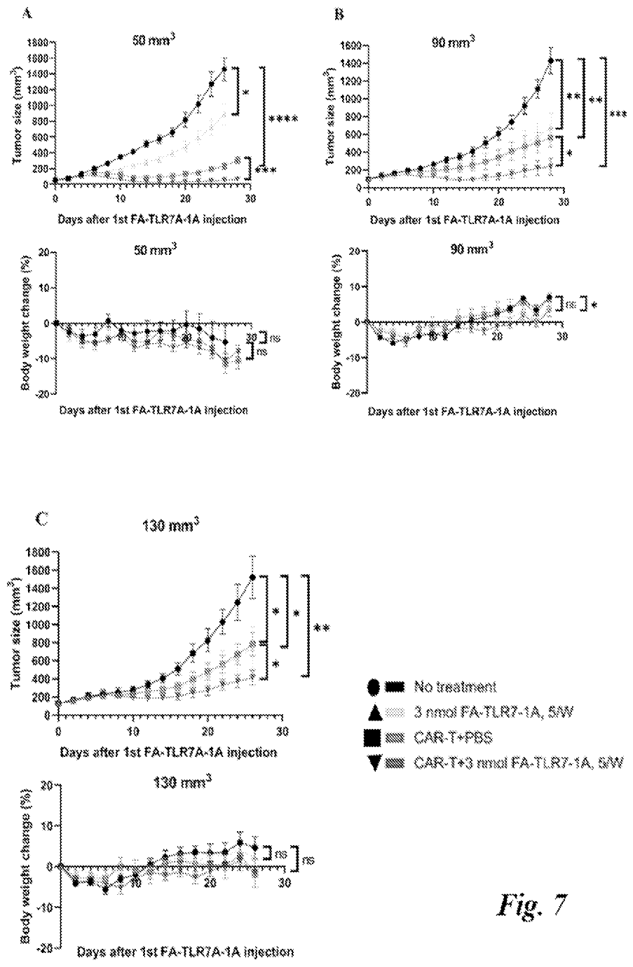

As shown in FIGS. 7A-7C, CAR-T cell efficacy decreases with enlarging tumor volume, while Compound 2 potency increases with tumor size.

### Example 8: Effects of Compound 2 on TAMs and MDSCs in the TME

It was next examined if the immune cells in the solid TME changed to a more tumoricidal phenotype (i.e., if the TME shifted to a more inflammatory state following administration of the combination therapies described herein). For this purpose, all tumor-bearing mice remaining after termination of the above-described investigations were euthanized and the tumors were isolated and digested with a Tumor Dissociation Kit (Miltenyi Biotec, Bergisch Gladbach, DE). The digested tumor cells were resuspended and stained with Zombie Violet (Biolegend, San Diego, CA) and antibodies for flow cytometry analysis.

To characterize the polarization states of the TAMs and MDSCs (i.e., the M1/M2 ratios of macrophages inside the tumors), digested cells were first stained with anti-mouse F4/80-APC and washed with PBS two times. The stained cells were then fixed and permeabilized with Cyto-Fast™ Fix/Perm Buffer Set (Biolegend, San Diego, CA). The cells were then incubated with the anti-mouse arginase-1-PE antibody and the anti-mouse iNOS-APC-eFluor 780 (eBioscience, Inc., San Diego, CA) for 20 minutes at room temperature in the dark. All samples were then washed two times with 1 mL PBS and analyzed by flow cytometry.

To check the percentages of MDSCs inside the tumor, digested cells were stained with anti-mouse CD11 b-APC-eFluor 780 (eBioscience, Inc., San Diego CA) and anti-Gr-1-Alexa-595 (BioLegend, San Diego, CA) and then washed two times with PBS. The splenocytes were isolated by gently pressing the spleens through the 70 μm cell strainer using 10 ml syringe plungers. The isolated cells were then stained with Zombie Violet and antibodies for flow cytometry analysis. All samples were incubated with anti-mouse CD16/CD32 (BioLegend, San Diego, CA) for 20 minutes on ice before being stained with antibodies.

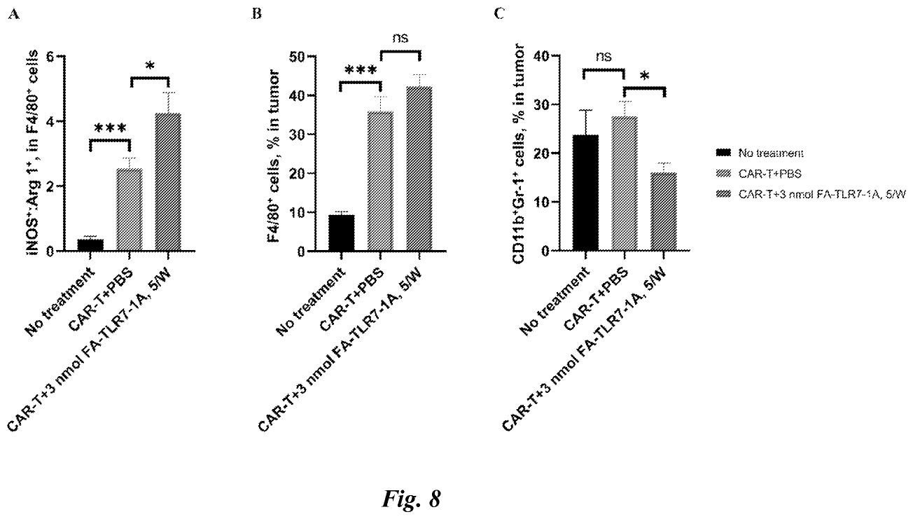

As shown in FIGS. 8A-8C, treatment with the combination hereof comprising Compound 2 doubled the ratio of M1:M2 macrophages as measured by iNOS:Arginase 1 ratio. Additionally, the same combination therapy roughly halved the numbers of MDSCs in each tumor mass. These results support that the targeted stimulation of TAMs and MDSCs shifted the TME myeloid cells to a more tumor-suppressing phenotype following treatment with the combination therapy described herein.

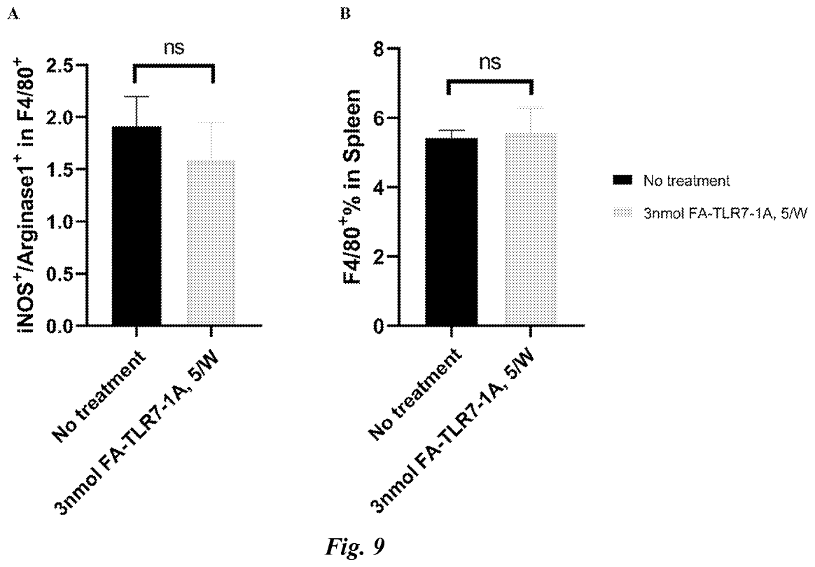

Further, to test the specificity of the delivery of Compound 2, the impact of the injection of Compound 2 on macrophages in spleens was examined. The splenic macrophages were previously reported to be FR⁻, and, consistent with this, the injection of Compound 2 did not have a significant impact on TAMs in the splenic macrophages as shown in FIGS. 9A-9B (i.e. the systemic administration of the FA-TLR7 agonist did not have a significant impact on the M1/M2 ratio or percentage of splenic macrophages). Accordingly, these results support that the impact of Compound 2 is specific to FR⁺ tumor-associated myeloid cells, and the systemic administration of Compound 2 is accurate and safe.

### Example 9: Effects of FA-TLR7-la on T Cells and CAR-T Cells in Solid Tumors

The effect on the T cells or CAR-T cells in the tumor following administration of the combination therapies hereof was assessed. The digested tumor cells were resuspended and stained with Zombie Violet (BioLegend, San Diego, CA) and antibodies for flow cytometry analysis. To check the number of infiltrated T cells and activated T cells inside the tumors, digested cells stained with anti-mouse CD3-APC (Invitrogen, Waltham, MA) with or without anti-mouse CD25-PE (BioLegend, San Diego, CA) or anti-mouse CD69-PE (BioLegend, San Diego, CA) were incubated on ice for 20 minutes and washed with PBS twice. All samples were incubated with anti-mouse CD16/CD32 for 20 minutes on ice before being stained with antibodies.

To verify the number of infiltrated CAR-T cells and activated CAR-T cells inside the tumors, digested cells were stained with an anti-rat IgG F(ab′)₂ Fragment conjugated with Alexa Fluor 594 for 30 minutes on ice and washed twice with PBS before adding anti-mouse CD16/CD32 and other antibodies.

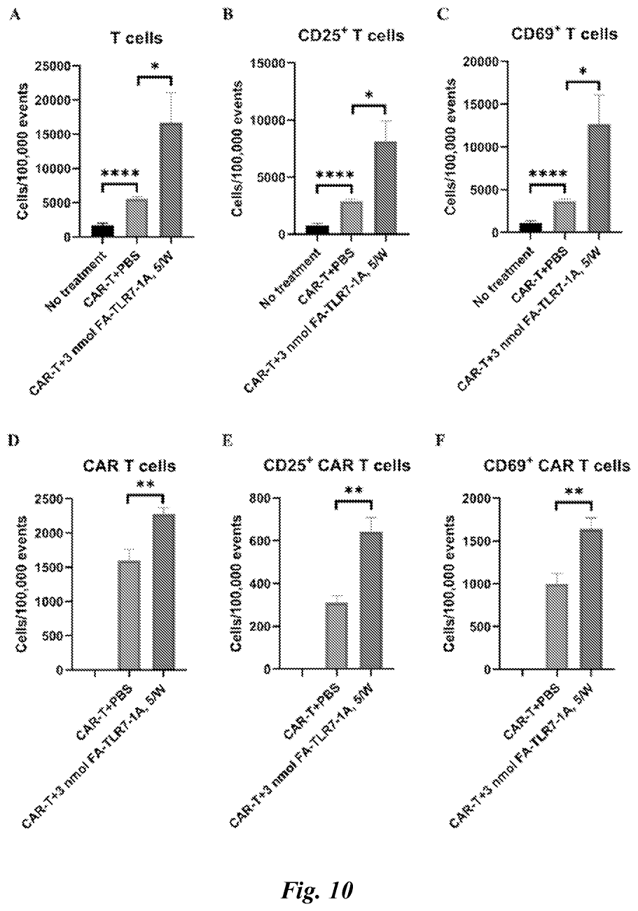

As shown in FIGS. 10A-10C, a significant impact on the number of infiltrated total T cells or activated T cells (e.g., CD3⁺ T cells) inside the tumors was not observed with Compound 2 treatment alone. However, treatment of Compound 2 significantly increased the number of infiltrated total and activated T cells and CAR-T cells inside the tumors when administered in combination with CAR-T cells (see FIGS. 10D and 10E).

For mice that did not receive a CAR-T cell treatment, the tumor sizes were too large at the end of the study. Therefore, while treatment with Compound 2 alone did increase the M1/M2 ratio as compared to the control treatment (no treatment), administration of the compound alone may not be sufficient to reprogram the immunosuppressive TME and further increase the activated T cells in a therapeutically significant manner. For the groups treated with anti-CD19 CAR-T cells, there were more activated CAR-T cells (indicated in FIGS. 10B and 10E by the increased number of CD25⁺ CAR-T cells, which is an activation marker, as well as in FIGS. 10C and 10F by the increased number of CD69⁺ CAR-T cells, which is a second T cell activation maker) because the immunosuppressive TME was changed by both the killing of 4T1-mCD19 cancer cells with the CAR-T cells and the repolarization of the FR⁺ TAMs and MDSCs by Compound 2.

Accordingly, the data support that administration of the combination therapies can improve the ML:M2 polarization ratio within the TME, reduce the number of immunosuppressive MDSCs in the tumor, and enhance CAR-T cell infiltration into the solid tumor.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result).

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

The terms and expressions, which have been employed, are used as terms of description and not of limitation. In this regard, where certain terms are defined, described, or discussed in various places in the “Detailed Description,” all such definitions, descriptions, and discussions are intended to be attributed to such terms. There also is no intention in the use of such terms and expressions of excluding any equivalents of the features shown and described or portions thereof “Conjugate” and “compound” may be used interchangeably herein.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered to be within the scope of the invention as claimed herein.

It is therefore intended that this description and the appended claims will encompass, all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

1. A combination cancer therapy comprising:
(a) at least one small molecule drug conjugate (SMDC) comprising a drug moiety conjugated to a ligand, wherein the ligand is specific to a receptor overexpressed on an immunosuppressive cell or a cancer cell, and
(b) chimeric antigen receptor (CAR)-expressing cytotoxic lymphocytes;
wherein the combination comprises a first amount of (a) and a second amount of (b), which together are effective to treat cancer.

2. The combination cancer therapy of claim 1, wherein the ligand comprises a folate receptor binding ligand or a fibroblast activation protein (FAP) ligand; and/or
each CAR is a fusion protein comprising a recognition region, a co-stimulation domain, and an activation signaling domain, and wherein the CAR binds a cell-surface antigen on an immunosuppressive cell or a cancer cell with specificity.

3. (canceled)

4. The combination cancer therapy of claim 1, wherein the drug moiety and the ligand are conjugated via a linker.

5. The combination cancer therapy of claim 1, wherein the drug moiety in at least one SMDC is an agonist of a pattern recognition receptor located in the endosome or the cytoplasm of a cell.

6. The combination cancer therapy of claim 1, wherein the drug moiety in at least one SMDC is selected from the group consisting of an agonist of a toll-like receptor (TLR), an agonist of a phosphoinositide 3-kinase inhibitor (PI3Ki), an agonist of a stimulator of an interferon gene (STING), an agonist of a nucleotide-binding oligomerization domain (NOD)-like receptor (NLR), an agonist of a retinoic acid-inducible gene-I (RIG-I)-like receptor (RLR), an agonist of an absent in melanoma 2 (AIM2)-like receptor (ALR), an agonist of a receptor for advanced glycation end products (RAGE), an agonist of a kinase of the Pelle/interleukin-1 (IL-1) receptor-associated kinase (IRAK) family, such as an IRAK-M inhibitor, an inhibitor of Src homology 2 domain-containing tyrosine phosphatase 1 and 2 (SUP1/2), an inhibitor of T cell protein tyrosine phosphatase (TC-PTP), an inhibitor of diacylglycerol kinase (DGK), an inhibitor of enhancer of zeste homolog 2 (EZH2), and an inhibitor of transforming growth factor beta (TGFβ).

7. The combination cancer therapy of claim 1, wherein the drug moiety in at least one SMDC is an agonist of TLR, a NF_{K}βactivator, an I_{K}β kinase inhibitor, or fluorescein isothiocyanate (FITC).

8-11. (canceled)

12. The combination cancer therapy of claim 4, wherein the linker comprises a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol.

13. The combination cancer therapy of claim 4, wherein the linker is (PEG)₃.

14-15. (canceled)

16. The combination cancer therapy of claim 1, wherein the SMDC is a folate-TLR7 agonist, a releasable form of a folate-(PEG)₃-TLR7 agonist, or a non-releasable form of a folate-(PEG)₃-TLR7 agonist.

17. (canceled)

18. The combination cancer therapy of claim 2, wherein the recognition region is a single chain variable fragment (scFv) of an antibody that binds to a cell-surface antigen with high specificity.

19. The combination cancer therapy of claim 18, wherein the cell-surface antigen is CD19.

20. The combination cancer therapy of claim 2, wherein the co-stimulation domain is CD28, CD137 (4-1BB), CD134 (OX40), CD2, or CD278 (ICOS); and/or
the activation signaling domain is a T cell CD3 chain or a Fc receptor γ.

21. (canceled)

22. The combination cancer therapy of claim 2, wherein:
the recognition region is a scFv region of an anti-FITC antibody, the co-stimulation domain is CD28, and the activation signaling domain is a T cell CD3ζ chain;
the recognition region is a scFv region of an anti-CD19 antibody, the co-stimulation domain is CD137 (4-1BB), and the activation signaling domain is a T cell CD3ζ chain; or
the recognition region is a scFv region of an anti-CD19 antibody, the co-stimulation domain is CD28, and the activation signaling domain is a T cell CD3ζ chain.

23-24. (canceled)

25. The combination cancer therapy of claim 1, wherein the cytotoxic lymphocytes are cytotoxic T cells, natural killer (NK) cells, lymphokine-activated killer (LAK) cells, or a combination of two or more of the foregoing.

26. A method of treating a subject for cancer in need thereof comprising administering the combination cancer therapy of claim 1 to the patient, whereupon the patient is treated for cancer.

27-29. (canceled)

30. The method of claim 26, wherein the cancer is a solid tumor cancer and further comprising imaging the solid tumor cancer prior to or during administering the combination cancer therapy.

31. The method of claim 26, wherein the cancer is a folate receptor expressing cancer.

32. The method of claim 26, wherein administering the combination cancer therapy further comprises administering a first therapeutically effective amount of the at least one SMDC and a second therapeutically effective amount of the CAR-expressing cytotoxic lymphocytes.

33. The method of claim 26, wherein one or both of the at least one SMDC and the CAR-expressing cytotoxic lymphocytes is administered to the subject via a mode of administration selected from the group consisting of intravenously, intramuscularly, intraperitoneally, and subcutaneously, wherein a mode of administration of the at least one SMDC is independent of a mode of administration of the CAR-expressing cytotoxic lymphocytes.

34-38. (canceled)

39. The method of claim 31, wherein the cancer is a folate receptor α-expressing cancer or a folate receptor β-expressing cancer.

40. The method of claim 32, wherein the first therapeutically effective amount of the at least one SMDC and the second therapeutically effective amount of the CAR-expressing cytotoxic lymphocytes are administered simultaneously or sequentially, in either order.

41. The method of claim 26, wherein administering the combination cancer therapy increases an amount of myeloid cells exhibiting an immune-stimulating phenotype in a tumor microenvironment (TME) of the subject as compared to an amount of myeloid cells exhibiting an immunosuppressive phenotype in the TME.

42. The method of claim 26, wherein administering the combination cancer therapy reduces an amount of myeloid-derived suppressor cells present within a TME of the subject.

43. A combination cancer therapy comprising:
a first pharmaceutical composition comprising at least one SMDC comprising a drug moiety or pharmaceutically acceptable salt thereof conjugated to a ligand, wherein the ligand is specific to a receptor overexpressed on an immunosuppressive cell or a cancer cell; and
a second pharmaceutical composition comprising CAR-expressing cytotoxic lymphocytes;
wherein the combination comprises a first amount of the first pharmaceutical composition and a second amount of the second pharmaceutical composition.

44-45. (canceled)
